(19) United States
(12) Patent Application Publication
(10) Pub. No.: US 2024/0272143 A1
(43) Pub. Date: Aug. 15, 2024

(54) RELEASE ASSAY FOR DETERMINING POTENCY OF SELF-AMPLIFYING RNA DRUG PRODUCT AND METHODS FOR USING

(71) Applicant: GLAXOSMITHKLINE BIOLOGICALS SA, Rixensart (BE)

(72) Inventors: Qiongman KONG, Rockville, MD (US); Xianzhi ZHOU, Rockville, MD (US); Varnika ROY, Rockville, MD (US)

(73) Assignee: GLAXOSMITHKLINE BIOLOGICALS SA, Rixensart (BE)

(21) Appl. No.: 18/567,056

(22) PCT Filed: Jun. 8, 2022
(86) PCT No.: PCT/IB2022/055356

§ 371 Date: Dec. 5, 2023

Related U.S. Application Data
(60) Provisional application No. 63/208,714, filed on Jun. 9, 2021.

(51) Int. Cl. C12Q 1/6804; G01N 15/01; G01N 15/10; G01N 15/14

(52) U.S. Cl. CPC C12Q 1/6804; G01N 15/01; G01N 15/14; G01N 2015/1006

(57) ABSTRACT

A potency release assay for measuring the potency of drug product composition comprising self-amplifying mRNA (SAM) that encodes at least one immunogenic polypeptide or at least one therapeutic peptide and a non-viral delivery system is described. In one embodiment the drug product is a vaccine comprising SAM and a non-viral delivery system such as SAM/lipid nanoparticle (LNP) delivery system, a Cationic Nanoemulsion (CNE) delivery system, or another SAM delivery system. It is demonstrated that the potency of a SAM drug product can be assessed in an in vitro system, at the RNA amplification stage (agnostic assay), by measuring the amount of double-stranded RNA (dsRNA) in cells which have been transfected with the SAM in the drug product. Thus, dsRNA can be used as a surrogate endpoint for potency. It is demonstrated that there is a very high correlation between total dsRNA in a cell culture transfected with the SAM and the potency of the SAM based drug product.

### CROSS-REFERENCE TO RELATED APPLICATION

This application is related to and claims priority to U.S. Provisional Application No. 63/208,714 filed on Jun. 9, 2021, the entire contents of which is hereby incorporated by reference.

### FIELD OF THE INVENTION

The present invention relates to a release assay for measuring the potency of a Self-Amplifying mRNA (SAM) drug product.

### BACKGROUND OF THE INVENTION

Vaccine development for newly discovered and existing viruses is important for public health. It is necessary for a vaccine, and other biological products, to be produced by Good Manufacturing Practices (GMP) facilities before being administered to a human patient, either for human clinical trials or for distribution to patients after FDA approval. During the FDA approval process, and also during manufacturing of individual lots of mRNA vaccines after FDA approval of the manufacturing protocols, it is necessary to establish that each lot of the vaccine meets stringent requirements for sterility, identity, purity and potency. See 21 C.F.R. §§ 610.1 and 610.2(a) and (b) and Jackson et al., “The promise of mRNA vaccines: a biotech and industrial perspective,” Vaccines (2020), Vol. 5, No. 11. Suitable pre-release testing may include a potency release assay.

One way of testing the potency of an mRNA construct in a research and development setting is to perform animal tests on the vaccine construct. For example, mRNA can be dispersed in a cationic nanoemulsion (CNE) to determine possible suitability for use in a vaccine. See Luisi et al., “Development of a potent Zika virus vaccine using self-amplifying messenger RNA” Sci. Adv. (2020), Vol. 6, No. 32, eaba5068. Luisi et al. describes transfection of baby hamster kidney (BHK) cells by use of electroporation with RNA constructs that encode a Zika virus viral antigen and subsequent measurement of the expression of the viral antigen using a pan-flavivirus E-specific monoclonal antibody. Maruggi et al. also describe introduction of a construct into cells by use of electroporation and subsequent measurement of antigen expression. See Maruggi et al., “mRNA as a Transformative Technology for Vaccine Development to Control Infectious Diseases,” Mol. Ther. (2019) April 10; 27(4): 757-772, which states that “A potency test is used to verify the ability of the mRNA to be translated into a desired protein product after delivery into target cells.” In previously reported assays to evaluate SAM constructs, it is necessary to have a diagnostic antibody for each different vaccine under development. See also Kong et al., “In-vitro Potency Assay for SAM RNA Vaccines,” CASSS DC Discussion Group (Jun. 7, 2018). In the Luisi et al, Maruggi et al. and Kong et al. assays, it is necessary to use a primary antibody specific to the immunogenic antigen of interest and to directly measure antigen expression at the protein level to determine potency. For each new antigen of interest, a new antigen expression assay is developed, requiring antibody screening, assay development, qualification, and the like.

In summary, assays for measuring possibly efficacy of a viral mRNA construct in the context of vaccine development are cumbersome, time consuming and/or expensive as such assays involve the use of antibodies specific to the expressed antigen, use of animals and/or use of techniques such as electroporation to introduce the RNA into cells in a cell culture.

### SUMMARY OF THE INVENTION

The present invention is directed to potency release assay for measuring the potency of drug product composition comprising SAM that encodes at least one immunogenic polypeptide or at least one therapeutic peptide and a non-viral delivery system. In one embodiment the drug product is a vaccine comprising SAM and a delivery system such as SAM/lipid nanoparticle (LNP) delivery system, a CNE delivery system, or another SAM delivery system. The present invention is based, in part, on the surprising discovery that the potency of a SAM drug product can be assessed in an in vitro system, at the RNA amplification stage, by measuring the amount of double-stranded RNA (dsRNA) in cells which have been transfected with the SAM in the drug product. This invention is an antigen agnostic assay that does not rely on the detection of specific antigen(s) after drug product delivery. Thus, dsRNA can be used as a surrogate endpoint for potency.

In one embodiment, the present invention comprises a cell-based in vitro potency assay that can characterize the potency of a SAM vaccine composition that comprises SAM and a delivery system. Products that can be tested in accordance with the present invention include, but are not limited to, products such as SAM/LNP and SAM/CNE vaccine products. The ability of a SAM delivery system to deliver SAM to cells (which in affects potency) can be impacted by (1) the non-viral delivery efficiency of SAM to the cells in an assay, and (2) the ability of SAM to amplify and form dsRNA in the cells, which cells then expresses the target antigen that is encoded by the SAM.

It has been surprisingly discovered that there is a high, reproducible correlation between the accumulation of detectable dsRNA in host cells at the RNA amplification stage (and thus the amount of antigen produced) and the ability of the SAM drug product to elicit the expression of a SAM encoded protein in a cell. Because of the high correlation that has been discovered by the inventors, it is not necessary to use a more expensive in vivo assay (for example an animal based potency assay) or an antigen expression assay to confirm the potency of the SAM vaccine product before releasing.

In one embodiment, the potency release assay measures the accumulation of dsRNA that is generated in cells which have been transfected with a SAM vaccine by using a technique that measures total dsRNA, for example a technique that uses a dsRNA specific antibody. It is not necessary to measure the amount of dsRNA in terms of nanograms, micrograms or milligrams per unit volume. Rather, the amount of dsRNA can be measured, for example, by determining the percentage of dsRNA positive cells in a cell culture assay. The percentage of dsRNA positive cells can be determined by any available method, but flow cytometer analysis is a suitable method.

The method of the invention can be used as a platform assay for various SAM Drug Products as it does not require use of an antibody that is specific for the antigen that is expressed by SAM in the cells. More specifically, in one embodiment of the present invention, it is not necessary to perform a further step of performing an antigen expression potency assay which requires a specific assay for each antigen used in each different drug product. Thus, the invention allows the use of an assay for measuring dsRNA that can be a platform assay that enables fast release of new drug products for emerging new pathogens or treatments and/or that can be used to test individual batches or lots of known drug products such as vaccines.

The in vitro dsRNA potency assay for a drug product, such as a SAM/LNP vaccine, can harness the cytosolic machinery of mammalian cells, such as BHK-21 cells or C2C12-Mouse Muscle Cells, to evaluate the ability of SAM to replicate and cause production of dsRNA, which is an accurate indicator of the ability of the SAM to express antigen. One important advantage of the present invention over prior art techniques that are used in the research and development setting is that instead of using a commercial transfection kit or electroporation to facilitate entry of mRNA into cells, as taught, for example, in Magini et al., “Self-Amplifying mRNA Expressing Multiple Conserved Influenza Antigens Confer Protection against Homologous and Heterosubtypic Viral Challenge,” PLOS ONE (Aug. 15, 2016), Vol. 11, No. 8, e0161193, entry of the mRNA into cells in accordance with the present invention relies on the delivery system in the drug product (such as SAM vaccine or an RNA+LNP complex). Thus, the present invention uses the delivery system that is present in the drug product, that is prepared for ultimate use on mammals, usually humans, to deliver the SAM to the cells used in the potency release assay. Thus, transfection can be accomplished by incubating a vaccine (drug product), such as SAM/LNP DP, with suitably cultured cells to allow for transmembrane cell entry without any additional steps to facilitate transfection such as electroporation and/or without addition of any additional special chemicals or agents to facilitate entry of the SAM into the cells. In one embodiment of the invention, the delivery system present in the vaccine (drug product) can be solely relied on, if desired, to facility entry of SAM into the cells used in the potency release assay.

The present invention includes at least the following embodiments:
1. A release assay for determining the potency of a drug product composition comprising self-amplifying mRNA (SAM) that encodes at least one an immunogenic polypeptide or that encodes at least one therapeutic polypeptide and a non-viral delivery system, said method comprising the steps of:
incubating said drug product composition with cells in vitro; and
detecting double-stranded RNA (dsRNA) in the cells.
2. The release assay of embodiment 1, wherein said SAM encodes at least one immunogenic polypeptide.
3. The release assay of embodiment 1 or 2, wherein said non-viral delivery system comprises lipid nanoparticles.
4. The release assay of any preceding embodiment, wherein said detecting step detects a signal that correlates to the amount of dsRNA in said cells.
5. The release assay of any preceding embodiment, wherein said detecting step comprises contacting said incubated cells with an antibody that binds dsRNA.
6. The release assay of embodiment 5, wherein said antibody that is contacted with said cells is a labeled antibody that specifically binds to dsRNA.
7. The release assay of any preceding embodiment, wherein said detecting step comprises determining the percentage of said cells that test positive in an assay that measures dsRNA in individual cells.
8. The release assay of embodiment 7, wherein the percentage of dsRNA positive cells is determined by flow cytometry analysis.
9. The release assay of any preceding embodiment, wherein said non-viral delivery system comprises liposomes.
10. The release assay of embodiment 7 or 8, wherein an anti-dsRNA antibody is used to bind to dsRNA in the cytoplasm of said cells and determining the percentage of dsRNA positive cells is performed by flow cytometry analysis.
11. The release assay of any preceding embodiment, further comprising the steps of comparing the results of said detecting step with predetermined results to determine if the results of said detecting step fall within pre-determined potency release standards and then releasing the drug product composition if the results of said detecting step fall within said predetermined release standards and not releasing the drug product if the results of said detecting step do not fall within said predetermined potency release standards.
12. The release assay of embodiment 11, wherein said method is performed on a sample from a lot of said drug product and the lot is not subjected to an assay that measures expression of said polypeptide before releasing the lot.
13. The release assay of any preceding embodiment, wherein said delivery system comprises liposomes that encapsulate SAM molecules.
14. The release assay of any preceding embodiment, wherein said delivery system comprises a lipid nanoparticle/SAM delivery system where said SAM molecules encode a polypeptide that elicits an immune response against at least one antigen present in a virus selected from the group consisting of rabies virus, HSV, CMV and COVID.
15. The release assay of any preceding embodiment, wherein said cells are an established mammalian cell line.
16. The release assay of embodiment 14, wherein said cells are a BHK cell line or a mouse muscle cell line.
17. The release assay of embodiment 15, wherein said cells are BHK-12 cells.
18. A release assay for determining the potency of a drug product composition comprising self-amplifying mRNA (SAM) that encodes at least one an immunogenic polypeptide and a non-viral delivery system, said assay comprising the steps of:
   incubating said drug product composition with cells in vitro;
   contacting said incubated cells with a reagent that measures dsRNA; and
   determining the percentage of said cells that test positive in an assay that measures dsRNA in individual cells.
19. A release assay for checking the potency of a drug product composition comprising self-amplifying mRNA (SAM) that encodes at least one an immunogenic polypeptide and a non-viral delivery system, said assay comprising the steps of:
   incubating said drug product composition with cells in vitro;
   contacting said incubated cells with a reagent that measures dsRNA;
   determining the percentage of said cells that test positive in an assay that measures dsRNA in individual cells; and checking that the percentage of dsRNA positive cells falls within a predetermined acceptable range.
20. The release assay of embodiment 18 or 19, wherein the reagent is an antibody that binds dsRNA, preferably a labelled antibody that binds dsRNA.
21. The release assay of embodiment 18, 19 or 20, wherein the percentage of dsRNA positive cells is determined by flow cytometry analysis.
22. A process of manufacturing a drug product comprising a composition comprising self-amplifying mRNA (SAM) that encodes at least one an immunogenic polypeptide or that encodes at least one therapeutic polypeptide and a non-viral delivery system for distribution to distributors, medical professionals or patients, the process comprising the steps of:
(a) manufacturing said drug product; and
(b) determining the potency of said drug product by the method of any of the preceding embodiments.
23. The process of embodiment 22, wherein said method is performed on a sample from a lot of said pharmaceutical product and the lot is not subjected to an assay that measures expression of said polypeptide before releasing the lot.
24. The process of embodiment 22 or 23, further comprising the steps of comparing the results of said detecting step with predetermined results to determine if the results of said detecting step fall within pre-determined potency release standards and then releasing the drug product if the results of said detecting step fall within said predetermined potency release standards and not releasing the drug product if the results of said detecting step do not fall within said predetermined potency release standards.

### BRIEF DESCRIPTION OF THE DRAWINGS

FIG. 1 is a flow chart to showing how an embodiment of the release assay fits in with the overall vaccine production and release process;

FIG. 2 illustrates an example of a first gate (P1) which plots SSC-A vs. FSC-A and which excludes cellular debris;

FIG. 3 illustrates an example of a second gate (P2) which plots FSC-H vs. FSC-A and which excludes doublets;

FIGS. 4A and 4B illustrate the boundary of a third gate (P3), which plots SSC-A vs APC-A (FIG. 4A) or GFP-A vs. APC-A (FIG. 4B), which rests between the negative population (cells to the left) and the positive population (cells to the right).

FIGS. 5A and 5B are graphs showing the correlation between the amount of dsRNA (FIG. 5A) and gB antigen (FIG. 5B) in the same target positive cells (detected via flow-cytometry characterization assay) at various SAM doses for three different lots of SAM CMV drug product encoding CMV Ag1 in a lipid nanoparticle delivery system. The percentage of target positive cells detected for dsRNA (FIG. 5A) at specific SAM-RNA doses is similar to the percentage of target positive cells detected for the gB antigen (FIG. 5B).

FIGS. 6A and 6B are graphs showing the correlation between the drug potency on dsRNA expression and the drug potency on gB antigen expression. The potency on either dsRNA expression or gB antigen expression are shown by comparing RP to standard by Global Fit to the % of gB⁺ cells after SAM CMV gB LNP treatment. FIG. 6C is a graph showing the direct RP correlation between gB and dsRNA detection by comparing relative potency to standard by % of gB⁺ cells to relative potency to standard by % of dsRNA⁺ cells.

FIGS. 7A and 7B show the impact of SAM structural integrity on SAM-Rabies/LNP potency by comparing IVRP (in vitro relative potency) to a reference standard, which is IVRP of full-length rabies/LNP drug product (%). (%). The correlation between dsRNA (FIG. 7B) and Rabies antigen (FIG. 7A) can be seen in the similar pattern of expression after drug product delivery.

FIGS. 8A,8B and 8C are graphs showing the correlation between antigen (% of antigen positive cells) and dsRNA (% of dsRNA-positive cells) expression levels in the evaluation of HSV/LNP lot-to-lot drug product variation, wherein the antigen measured in FIG. 8A is HSV gE antigen; the antigen measured in FIG. 8B is HSV gI antigen; and the target measured in FIG. 8C is dsRNA. dsRNA (FIG. 8C) and specific antigens (FIGS. 8A and 8B) follow similar patterns of expression at specific SAM-RNA doses and there is a high level of potency correlation between dsRNA and antigen expression levels.

FIGS. 9A-9D are charts showing the correlation between antigen expression (% of antigen-positive cells) and dsRNA (% of dsRNA-positive cells) expression levels in the evaluation of HSV/LNP product stability, wherein assay results (in terms of % positive cells) are plotted against SAM/RNA dose (ng), wherein the assays in each Figure are measurement of gE antigen (Ag1) (FIG. 9A) and gI antigen (Ag2) (FIG. 9B) for Tox batch drug product tested at T0 time point (TO Tox) and after storage for 2 weeks at 25° C. (2 weeks at 25° C. Tox), and measurement of dsRNA for 2 weeks at 25° C. Tox (FIG. 9C), and T0 Tox (FIG. 9D). dsRNA (FIGS. 9C and 9D) and specific antigens (FIGS. 9A and 9B) follow similar patterns of expression at specific SAM-RNA dose, s delivered immediately or after a 2-week storage, and there is a high level of potency correlation between dsRNA and antigen expression levels at specific time points.

FIG. 10 is a graph showing the relative potency correlation between dsRNA and antigen expression for various antigens by comparing relative potency to standard by % of antigen positive cells to relative potency to standard by % of dsRNA positive cells.

### DETAILED DESCRIPTION OF THE INVENTION

As discussed above, the present invention relates to an assay for measuring the potency of a SAM vaccine. Some of the preferred or alternative features of the invention will be described in more detail below.

### Timing of the Assay in Relation to Manufacturing Steps

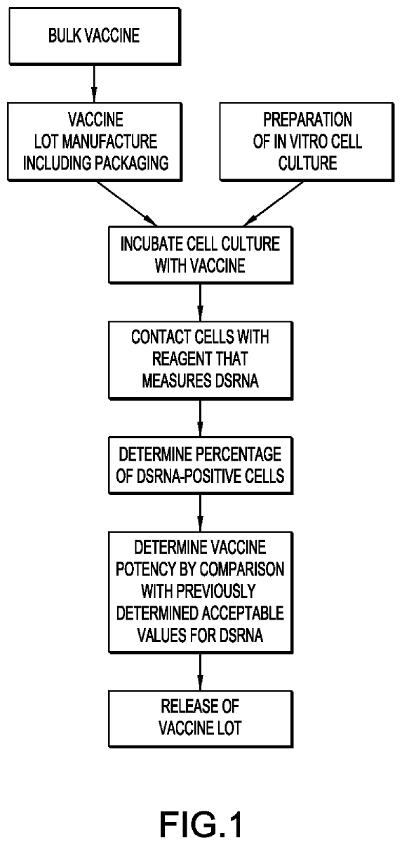

The method of the present invention is a release assay for any type of SAM Drug Product. As shown very generally in FIG. 1, and as described in more detail below, prior to performing the assay, a vaccine sample to be tested and a cell culture are separately prepared. Then, a release assay is conducted which include steps of incubating the cell culture with a SAM Drug Product (such as a vaccine drug product) sample, contacting the cells with a reagent that indicates the presence of dsRNA and determining the amount of dsRNA in the Drug Product sample. After the amount of dsRNA is determined, the potency of the Drug Product sample it determined by comparing the potency value obtained in the assay with previously determined potency values (based on amount of dsRNA). If the potency value determined by the assay is within predetermined acceptable potency release standards, the Drug Product lot can be released for distribution, assuming of course that any other necessary pre-requisites or standards for release are met.

The potency release assay can be performed on a Drug Product sample (such as a vaccine sample) from a lot of Drug Product samples prepared under Good Manufacturing Practices (GMP) but which Drug Product lot has not yet been released for distribution to distributors, medical professionals or patients. Alternatively, the Drug Product sample could be obtained from a bulk Drug Substance, before the bulk Drug Substance is provided for unit packaging and before subsequent distribution to distributors, medical professionals, or patients. In the case of a SAM/LNP vaccine or any other Drug Substance that is completely formulated before the vaccine or Drug Substance will be distributed, the timing of the assay can be as described above where a vaccine or Drug Substance sample being tested has already been formulated. In the case of a vaccine where two or more vaccine components are maintained in separate containers as part of a kit or packaged combination during distribution, and where the two or more vaccine components are not combined until shortly before use, the assay may be performed on a vaccine kit or approved Drug Product. In such a situation, the vaccine composition is prepared according to approved instructions associated with the kit and the release assay is then performed on the resulting vaccine composition. Such a vaccine kit, where all the components of the vaccine are not yet combined with each other, may be used for a CNE/SAM vaccine that includes the CNE and the SAM, each packaged separately, wherein the CNE and SAM are mixed with each other according to packaging instructions just before performing the assay on the vaccine composition. In such a situation, the vaccine kit will include the CNE and SAM, each packaged separately, and the packaged CNE and SAM will be packaged in another package, such as a box, envelope or other package, together with instructions for making and administering the vaccine composition. The assay of the invention may be performed on one or more Drug Product kits or vaccine kits in a particular lot before the rest of the kits in the lot are released for distribution.

### Preparing the Cells for the Assay

The cells in which the assay is performed are placed in a container with a suitable growth or incubation medium. Typically, a vial of cells will be removed from liquid nitrogen and thawed by standard techniques for subsequent cell culture. Cell culture medium will also be brought to a suitable temperature, such as room temperature or higher, as needed. The cells can then be mixed with the medium in a sterile tube to disperse the cells in the medium, usually at a temperature between room temperature and the anticipated incubation temperature. The cells are then seeded to a larger container (typically a sterile flask, but any other container such as a sterile culture dish can be used) in an amount sufficient to prepare a cell culture containing a predetermined percentage of viable cells, a predetermined confluency range, and/or a predetermined optimal growth phase. If an existing (non-frozen) cell culture is available, the cells from the existing culture within the preferred passage number can be prepared so that the cells have the predetermined desired characteristics for the subsequent assay. In preparation for the assay, the cells should be suspended in an assay medium.

The following predetermined characteristics, which work well for BHK-21 cells, may also work well for other cells:

| Characteristic | Preferred | Ideal |
|---|---|---|
| Viable cells | ≥85% | ≥90% |
| Confluency | 65-90% | 75-85% |
| Passage number for assay | ≥3; ≤25 | ≥3; ≤25 |

Although the above predetermined characteristics have been developed for BHK cells, and in particular BHK-21 cells, for a “platform assay” that can be used to test various mRNA vaccines, other predetermined characteristics and/or other predetermined values for the above characteristics can be set for any particular assay, depending on convenience, cell type vaccine type, etc. The most important point in setting predetermined characteristics of the cell culture used in the assay is that the cell culture should be prepared so when the assay is conducted on the cell culture, a meaningful potency value can be determined based on predetermined potency release standards, the predetermined release standards being established in previously performed assays that used cell cultures having the same or similar predetermined characteristics.

### Dilution of Cells for Delivery of SAM and Preparation of Cells for Assay

After cells with the above predetermined characteristics are prepared, the cells are grown to (or diluted, if necessary) to a pre-determined density range for transfection of the cells by the SAM (which is a component of the SAM the Drug Product). After cells of an appropriate density and other characteristics are prepared, cells may be introduced into a different container to which the vaccine composition will be added. Ideally, cells are introduced into multiple wells on a multi-well plate (such as a 96-well plate) so that various dilutions of the Drug Product composition can be added to different wells by techniques known in the art. Assay Medium is added as necessary to bring the volume in each well to the same predetermined level.

### Preparation of Vaccine Samples Containing SAM for Use in the Assay

The same Assay Medium can be used to prepare/dilute the cells for assay and to prepare/dilute the Drug Product for assay. Serial dilutions of the vaccine to be tested are prepared by diluting the vaccine with a diluent suitable for the assay, such as the same Assay Medium used to prepare the cells for testing. Ideally, enough dilutions of the Drug Product are prepared so that a full dose response curve can be generated, and this dose response curve can be compared with a dose response curve previously generated when setting potency release standards for the assay.

Introducing the SAM into the Cells and Incubation of the SAM with the Cells

The SAM in the Drug Product or vaccine sample is introduced into the cells to cause transfection of the cells and expression of the SAM in the cells by contacting the cells with the SAM Drug Product or vaccine sample (usually diluted with in an Assay Medium) and incubating the SAM Drug Product or vaccine sample with the cells in vitro for a sufficient amount of time to allow the SAM to enter the cells and replicate in the cytoplasm of the cells to produce dsRNA. When multi-well plates are used, such as when serial dilutions of Drug Product or vaccine are tested, different predetermined dilutions of the vaccine (or a control solution) can be added to different wells containing the cells.

Incubation should be conducted for a pre-determined time sufficient to allow delivery of the SAM to the cytosol by the delivery system in the Drug Product, such as LNPs in a Drug Product or vaccine sample, and then for a further time sufficient for the SAM to replicate and form dsRNA to be transcribed and translated into target antigen. dsRNA is measured during an RNA amplification stage, i.e., a stage where the SAM Drug Product is actively producing dsRNA in the cells. Although the target antigen(s) are not directly measured in the SAM release assay of the present invention, the dsRNA is ideally measured at a time when translation of the target antigen is actively occurring in the cell culture. The ideal incubation time will be determined for each type of cell/vaccine combination and/or any established protocol for a particular vaccine. Typically, the cells are contacted with the SAM Drug Product or vaccine on one day (Day 1) and the assay for dsRNA is conducted (or at least begun) on the next day (Day 2) or any other predetermined amount of time. Therefore, incubation may be conducted for a pre-determined time period such as 24 hours, which in the assay reported in the present application can be 12-36 hours, preferably 16-32 hours, more preferably 20-28 hours and most preferably 22-26 hours. As with other parts of the assay, the most important factor is that the incubation time is set to a pre-determined range so that meaningful results from the assay can be obtained to determine if the vaccine is ready for release.

Quantifying or Measuring the Amount of dsRNA

The procedures to measure the amount of dsRNA in this application measure “% positive cells.”

After a suitable predetermined incubation period, cells from the incubation step are prepared for determining the amount of dsRNA. When a multi-well plate is used, cells from each well are collected separately and maintained separately during dsRNA measurement. The amount of dsRNA can be measured by any technique that measures the amount of dsRNA. It is not necessary to detect the amount of dsRNA that specifically encodes the immunogenic polypeptide in the SAM Drug Product or vaccine; measurement of total dsRNA is sufficient. Thus, any convenient technique that measures total mRNA can be used and it is not necessary to use a technique that only measures dsRNA that is generated by replication of the SAM molecules in the vaccine. A preferred way to measure dsRNA is to use an antibody that is specific for dsRNA. After cells that have been contacted with the SAM vaccine are incubated for a sufficient period of time, the amount of dsRNA can be quantified by contacting the cells with the antibody that specifically reacts with dsRNA under conditions that allow the antibody to bind to the dsRNA. In one embodiment, the antibody is contacted with dsRNA when it is located inside the cells and the percentage of dsRNA positive cells in a sample of cells is determined. In order to prepare the cells for dsRNA measurement, the cells can be harvested, washed, fixed and permeabilized overnight to ready the cells for contact with labelled antibody which detects the presence of dsRNA. After the cells are permeabilized, the permeabilized cells are contacted with the dsRNA specific antibody and the labelled antibody enters the cells.

The “amount” of dsRNA need not be determined in terms of absolute amount of dsRNA. Rather, any reproducible technique can be used if it produces results that correlate to the amount of dsRNA in the cells, and which can be reproducibly compared with results obtained from a previously conducted similar technique that was used to create the potency release standards. A convenient technique is to determine the percentage of cells that test positive in antibody test using an antibody that specifically reacts with dsRNA. A test that counts “dsRNA positive cells” is possible. In order to determine which cells are positive for dsRNA, the anti-dsRNA antibody can be labeled with a suitable label so that the positive cells can be easily counted. The percentage of dsRNA positive cells can be determined by setting the assay conditions and setting the criteria for positive cells by techniques known in the art.

In one embodiment, a suitable labeled antibody can be prepared by incubating a primary unlabeled dsRNA-specific antibody (such as an anti-dsRNA mouse monoclonal antibody) with a labelled antibody that specifically reacts with the Fc portion of the dsRNA antibody to prepare a labeled dsRNA-specific antibody by techniques known in the art. For example, Zenon allophycocyanin-conjugated anti-mouse Fab fragment that binds to the Fc portion of the anti-dsRNA antibody can be used to prepare the labeled dsRNA specific antibody. The labeled Fab fragment binds to the Fc portion of the dsRNA antibody. Then, excessive unbound labeled Fab fragment is removed, for example, by mixing with nonspecific mouse IgG, which binds with the Fc portion of the dsRNA antibody. Any remaining bound antibody that is not fixed to the cells is then removed. The treated cells (many of which are labeled) should then be ready for analysis by flow cytometry.

Prior to contacting the labeled antibody with the cells, the cells are permeabilized so that the labeled antibody can enter the permeabilized cells and react with (bind to) the dsRNA present in the cytoplasm of the cells. Before being permeabilized, the cells are usually transferred to individual wells of a different plate that is more suitable for the next steps.

Positive cells and negative cells are then detected (counted) by any convenient technology such as Flow Cytometry, and the percentage of positive cells is determined.

The total amount of dsRNA can also be measured in other types of “units” and/or by use of different techniques such as an ELISA assay. When an ELISA assay is used, the above-described anti-dsRNA antibody can be used in the assay to bind to the dsRNA and then the amount of dsRNA can be measured by the ELISA assay. The most important point is that the procedures used in the release assay are the same as (or correlate highly with) the procedures used to set the potency release standards.

Analysis of the Data and Comparison with Pre-Determined Potency Release Standards

Data analysis is conducted by techniques known in the art. In the present application, data analysis was conducted using the commercially available software FlowJo™ Software obtained from FlowJo, LLC or equivalent software. Experiment related data files can be copied from the equipment build-in computer for data storage and future remote analysis by FlowJo or other equivalent software. When analyzing data performed by a flow cytometer, software supplied by the manufacturer of the flow cytometer can also be used if it has acceptable data analysis functions. Fine-tuning and/or adjustments of the equipment, such as gating, are performed as instructed by the manufacturer or by techniques known in the art in order to obtain reliable data. Data analysis is used to create potency release standards for any specific assay, based on different assay protocols, such as protocols that use different cells, different equipment, different media, different antibodies, different incubation conditions, different SAM molecules of interest, etc. An important factor in this regard is that it is preferred that a meaningful and suitable dose response curve is preferably generated. Data analysis is also used at the stage of performing the final release assay for each batch so it can be determined if the dsRNA measurements meet predetermined release standards.

### Cells for Use in the Method

Various different types of vertebrate cells are potentially suitable for use in accordance with the present invention. The main requirement of the cells is that the SAM in the Drug Product must be capable of transfecting the cells and reproducibly replicating within the cells to give meaningful measurements. Cells from mammals are particularly suitable. Ideally, the cells are immortal mammalian cell lines. BHK-21 cells were used in the experiments reported below, but other types of BHK cells or other types of mammalian cells such as C2C12 (ATCC CRL-1772) cells, which is a specific mouse muscle cell line also produces a good dose-response curve and therefore is also a suitable cell-line.

### Antibodies

If an antibody or antibodies are used to quantify the amount of dsRNA, any antibody that specifically recognizes dsRNA in cells during mRNA replication can be used. A monoclonal antibody is preferable. A suitable antibody will not recognize DNA or other forms of RNA (ssRNA, tRNA, rRNA), as explained further in Schonborn et al., “Monoclonal antibodies to double-stranded RNA as probes of RNA structure in crude nucleic acid extracts,” Nucleic Acids Research 1991) Vol. 19, No. 11, pp. 2993-3000 (1991). The antibody is preferably a monoclonal antibody such a J2 manufactured by Scicons. See, “Antibodies—Monoclonal anti-dsRNA J2 and anti-dsRNA K1, SCICONS J2 Tech. Spec. (2014) (https://scicons.eu/en/antibodies/j2/). This antibody has been proven to detect dsRNA intermediates of viruses as diverse as Hepatitis C virus, Dengue virus, rhinovirus, Chikungunya virus, rabies virus, polio virus, classic swine fever virus, Brome mosaic virus and others in cultured cells and also in paraffin-embedded histological samples. The J2 antibody recognizes dsRNA longer than 40 bp, regardless of the sequence and nucleotide composition. Antibodies K1 and K2, also provided by Scicons, may also be suitable. A suitable antibody for use in this invention will also detect dsRNA longer than 40 bp, regardless of the sequence, especially dsRNA of viral origin.

### SAM Molecules

SAM molecules present in a Drug Product that can be assayed for in accordance with the present invention can be prepared by methods known in the art. In general, SAM molecules contain at least mRNA that encodes an immunogenic polypeptide that elicits an immune response against infection with the associated pathogen and elements that allow the mRNA to self-replicate in vitro in a cell culture and to also replicate in vivo in an animal and/or human. The SAM molecules may encode a full-length viral antigen or an immunogenic fragment thereof. A SAM molecule typically comprises a cap structure (such as a Cap0 structure (a 7-methylguanosine connected to the 5′ nucleotide) or a Cap1 structure (containing further methylation at the 2′-OH position of the first nucleotide)), 5′ and 3′ untranslated regions (UTRs), an open-reading frame (OFR), and a 3′ poly(A) tail. The SAM molecule will also contain genetic replication machinery, usually derived from positive-stranded mRNA viruses, including alphaviruses. Generally, the ORF encoding native viral structural proteins is replaced by the selected transcript of interest, and the viral RNA-dependent RNA polymerase is retained to direct cytoplasmic amplification of the replicon construct. In some embodiments, the SAM molecule does not itself encode a transcript of interest, i.e., the ORF encoding native viral structural proteins is removed, but not replaced by any coding sequence. SAM molecules can have various lengths but they are typically 5,000-25,000 nucleotides long e.g. 8,000-15,000 nucleotides, or 9,000-12,000 nucleotides. In some embodiments, the SAM molecule contains subgenomic RNA sequences recognized by the viral RNA-dependent polymerase and a transcript of interest; the separate RNA molecule is then replicated by the viral RNA-dependent polymerase in trans.

Techniques for making viral antigen SAM constructs are described, for example, in the following references: Sharma et al. in WO 2017/208191 A1 (GLAXOSMITHKLINE BIOLOGICALS SA), entitled ZIKA VIRAL ANTIGEN CONSTRUCTS, which describes preparation of Zika virus SAM constructs; Geall et al., WO 2012/006377 A2 (NOVARTIS AG), entitled DELIVERY OF RNA TO TRIGGER MULTIPLE IMMUNE PATHWAYS, which describes that RNA with standard nucleotides delivered to cells triggers both an endosomal innate immunity receptor (e.g. TLR7) and also a cytoplasmic innate immunity receptor (e.g. a RNA helicase such as MDA5 or RIG-I); Geall et al., WO 2012/006372 (NOVARTIS AG), entitled DELIVERY OF RNA TO DIFFERENT CELL TYPES, which discloses delivery of RNA with standard nucleotides encoding an immunogen to non-immune cells at the site of delivery, and also to immune cells; and Geall et al., WO 2012/006369 (NOVARTIS AG), entitled IMMUNISATION OF LARGE MAMMALS WITH LOW DOSES OF RNA, which describes immune response in a large mammal, comprising administering to the mammal a dose of between 2 μg and 100 μg of a self-replicating RNA with standard nucleotides encoding an immunogen. The methods for preparing SAM constructs, as well as the methods of formulating the SAM constructs into a vaccine composition for administration to a subject are incorporated by reference into the present application.

The present invention is applicable to various types of SAM molecules that encode polypeptides that have a preventative, protective and/or therapeutic effect in a patient. The SAM molecules may encode a polypeptide that encodes a therapeutic or protective response against infections by various pathogens such as viral, bacterial and protozoan pathogens. In addition to SAM molecules that encode polypeptides that elicit a protective immune response, SAM molecules that encode antibodies, antibody fragments, single chain antibodies, polypeptides that may be lacking in a patient due to a genetic defect (such as in known enzyme replacement therapy techniques), etc. may be incorporated into the Drug Product.

The SAM molecules can be present in known and already approved Drug Products, such as vaccines, or Drug Products developed and/or approved in the future. Drug Products against infectious agents or organism, and in particular viruses that infect humans, are of great interest. In the context of Drug Products that are vaccines, the SAM molecule encodes one or more polypeptides (sometimes referred to as an antigen or immunogen) is usually one that elicit a protective immune response to one or more capsid proteins from HSV, CMV, Rabies, COVID, or one of the other viruses described below. Thus, in some embodiments, the immunogen elicits an immune response against one or more viral antigens disclosed in WO 2013/006378 (the relevant portions of which are incorporated by reference), including one or more of the following viruses or viral antigens:

Orthomyxovirus: Useful immunogens can be from an influenza A, B or C virus, such as the hemagglutinin, neuraminidase or matrix M2 proteins. Where the immunogen is an influenza A virus hemagglutinin it may be from any subtype e.g. H1, H2, H3, H4, H5, H6, H7, H8, H9, H10, HI 1, H12, H13, H14, H15 or H16.

Paramyxoviridae viruses: Viral immunogens include, but are not limited to, those derived from Pneumoviruses (e.g. respiratory syncytial virus, RSV), Rubulaviruses (e.g. mumps virus), Paramyxoviruses (e.g. parainfluenza virus), Metapneumoviruses and Morbilliviruses (e.g. measles).

Poxviridae: Viral immunogens include, but are not limited to, those derived from Orthopoxvirus such as Variola vera, including but not limited to, Variola major and Variola minor.

Picornavirus: Viral immunogens include, but are not limited to, those derived from Picornaviruses, such as Enteroviruses, Rhinoviruses, Heparnavirus, Cardioviruses and Aphthoviruses. In one embodiment, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.

Bunyavirus: Viral immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.

Heparnavirus: Viral immunogens include, but are not limited to, those derived from a Heparnavirus, such as hepatitis A virus (HAV).

Filovirus: Viral immunogens include, but are not limited to, those derived from a filovirus, such as an Ebola virus (including a Zaire, Ivory Coast, Reston or Sudan ebolavirus) or a Marburg virus.

Togavirus: Viral immunogens include, but are not limited to, those derived from a Togavirus, such as a Rubivirus, an Alphavirus, or an Arterivirus. This includes rubella virus. Flavivirus: Viral immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus.

Pestivirus: Viral immunogens include, but are not limited to, those derived from a Pestivirus, such as Bovine viral diarrhea (BVDV), Classical swine fever (CSFV) or Border disease (BDV).

Hepadnavirus: Viral immunogens include, but are not limited to, those derived from a Hepadnavirus, such as Hepatitis B virus. A composition can include hepatitis B virus surface antigen (HBsAg).

Other hepatitis viruses: A composition can include an immunogen from a hepatitis C virus, delta hepatitis virus, hepatitis E virus, or hepatitis G virus.

Rhabdovirus: Viral immunogens include, but are not limited to, those derived from a Rhabdovirus, such as a Lyssavirus {e.g. a Rabies virus) and Vesiculovirus (VSV).

Caliciviridae: Viral immunogens include, but are not limited to, those derived from Calciviridae, such as Norwalk virus (Norovirus), and Norwalk-like Viruses, such as Hawaii Virus and Snow Mountain Virus.

Coronavirus: Viral immunogens include, but are not limited to, those derived from a SARS coronavirus, COVID-19 (including various strains thereof), avian infectious bronchitis (IBV), Mouse hepatitis virus (MHV), and Porcine transmissible gastroenteritis virus (TGEV). The coronavirus immunogen may be a spike polypeptide. See, e.g., Wrapp et al. (2020) “Cryo-EM structure of the 2019-nCoV spike in the prefusion conformation.” Science, 367:1260-1263.

Retrovirus: Viral immunogens include, but are not limited to, those derived from an Oncovirus, a Lentivirus {e.g. HIV-1 or HIV-2) or a Spumavirus.

Reovirus: Viral immunogens include, but are not limited to, those derived from an Orthoreovirus, a Rotavirus, an Orbivirus, or a Coltivirus.

Parvovirus: Viral immunogens include, but are not limited to, those derived from Parvovirus B19.

Herpesvirus: Viral immunogens include, but are not limited to, those derived from a human herpesvirus, such as, by way of example only, Herpes Simplex Viruses (HSV) {e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).

Papovaviruses: Viral immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.

Adenovirus: Viral immunogens include those derived from adenovirus serotype 36 (Ad-36).

### SAM Delivery Systems

Various types of SAM delivery systems may be used in accordance with the present invention. The SAM molecules useful in the Drug Products of the assay of the present invention include any non-viral delivery system that delivers the SAM across the cell membrane of a subject to which it is administered. The delivery systems are non-viral delivery systems that do not contain virus particles. Although the particles are, they can be synthetic virion particles that do not comprise protein capsid. The SAM encoding an immunogen can be encapsulated by the delivery material, adsorbed to the delivery material or may form an emulsion with the delivery material.

Suitable non-virion delivery systems are described, for example, by Geall et al., in WO 2012/006376 A2 (NOVARTIS AG) entitled VIRION-LIKE PARTICLES FOR SELF-REPLICATING RNA MOLECULES. The entire contents of this reference relating to non-virion delivery systems is incorporated by reference herein. Lipid based delivery systems include, but are not limited to, lipid nanoparticle (LNP) delivery systems and cationic nanoemulsion (CNE) delivery systems. In this reference, particles are non-virion particles i.e., they are not a virion. Thus, the particle does not comprise a protein capsid. By avoiding the need to create a capsid particle, the invention does not require a packaging cell line, thus permitting easier up-scaling for commercial production and minimizing the risk that dangerous infectious viruses will inadvertently be produced. Instead of encapsulating RNA (SAM molecule in the context of the present invention) in a virion, particles are formed from a delivery material. Various materials are suitable for forming particles which can deliver RNA to a vertebrate cell in vivo. Two delivery materials of particular interest are (i) amphiphilic lipids which can form liposomes and (ii) non-toxic and biodegradable polymers which can form microparticles such as nanoparticles. Where delivery is by liposome, SAM should be encapsulated; where delivery is by polymeric microparticle, SAM can be encapsulated in or adsorbed on the microparticle. A third delivery material of interest is the particulate reaction product of a polymer, a crosslinker, a SAM, and a charged monomer.

Thus, one embodiment of a particle of the invention comprises a liposome encapsulating a SAM molecule which encodes an immunogen, whereas another embodiment comprises a polymeric microparticle encapsulating a SAM molecule which encodes an immunogen, and another embodiment comprises a polymeric microparticle on which a SAM molecule which encodes an immunogen is adsorbed. In all three cases the particles preferably are substantially spherical. In a fourth embodiment a particle of the invention comprises the particulate reaction product of a polymer, a crosslinker, a SAM molecule which encodes an immunogen, and a charged monomer.

SAM can be encapsulated within the particles (particularly if the particle is a liposome). This means that SAM inside the particles is (as in a natural virus) separated from any external medium (which is usually an aqueous environment) by the delivery material, and encapsulation has been found to protect SAM from RNase digestion. Encapsulation can take various forms. For example, in some embodiments (as in a unilamellar liposome) the delivery material forms a outer layer around an aqueous SAM-containing core, whereas in other embodiments (e.g. in molded particles) the delivery material forms a matrix within which SAM is embedded. The particles can include some external RNA (e.g. on the surface of the particles), but at least half of the RNA (and ideally all of it) is encapsulated. Encapsulation within liposomes in this context is distinct from, for instance, the lipid/RNA complexes disclosed in Johanning et al. (1995), Nucleic Acids Res 23:1495-1501.

SAM can be adsorbed to the particles (particularly if the particle is a polymeric microparticle). This means that RNA is not separated from any external medium by the delivery material, unlike the RNA genome of a natural virus. The particles can include some encapsulated SAM (e.g. in the core of a particle), but at least half of the RNA (and ideally all of it) is adsorbed.

Suitable delivery systems are also disclosed by Geall et al. in WO 2012/006378 (NOVARTIS AG) entitled LIPOSOMES WITH LIPIDS HAVING AN ADVANTAGEOUS PKA-VALUE FOR RNA DELIVERY, which discloses delivery of RNA using lipids having a pKa within a specific range. This reference is also incorporated by reference.

Nanoparticle based delivery systems are particularly useful in in the present invention. Nanoparticles are small particles (generally on the order of 1-1,000 nanometers) that are used as part of a delivery system for SAM molecules. Nanoparticles include, without limitation, LNPs, CNEs and polymer-based nanoparticles. Nanoparticles include at least the nanoparticles described by Zhao et al, Nanoparticle vaccines, Vaccine, 32 (2014) 327-337. The LNPs are associated with the SAM and the LNPs assist delivery of the SAM into the mammalian cells. The SAM molecules can be bound to the exterior of LNPs, can be encapsulated within the LNPs, embedded in the lipid portion of the LNPs and/or emulsified with the LNPs. In one embodiment, an effective amount of SAM molecules are encapsulated within the LNPs to form liposomes to deliver the SAMs into the target cells so that said SAMs can cause expression of an effective immunogenic amount of said immunogenic polypeptide. The lipids used to form lipid nanoparticles or a cationic nanoparticle emulsions usually comprise one or more cationic lipids and optionally other lipids. For example, a cationic nanoparticle emulsion (CNE) may comprise cationic lipids nanoparticles containing the SAM construct, vector, or self-replicating mRNA molecules in the form of and oil-in-water emulsion of lipid nanoparticles in an aqueous environment.

Anderluzzi et al, Investigating the Impact of Delivery System Design on the Efficacy of Self-Amplifying RNA Vaccines, Vaccines (Basel), Vol. 8, No. 2 (June 2020) describes the preparation of and various desirable attributes of certain cationic lipid-based delivery systems for SAM vaccines. In particular, the following four different types of platforms were investigated: (1) liposomes, (2) solid lipid nanoparticles (SLNs), (3) polymeric nanoparticles (NPs) and emulsions. All of these types of nanoparticles are useful in accordance with the present invention and the entire contents of this reference are incorporated by reference with respect to how to prepare the formulations and the properties of the formulations. Briefly, these formulations contained either the non-ionizable cationic lipid 1,2-dioleoyl-3-trimethylammonium-propane (DOTAP) or dimethyldioctadecylammonium bromide (DDA)

### Lipid Nanoparticle (LNP) and Microparticle Delivery Systems

LNP delivery systems and non-toxic biodegradable polymeric microparticles, and methods for their preparation are described in the following references: WO2012/006376 (LNP and microparticle delivery systems); Geall et al. (2012) PNAS USA. September 4; 109(36): 14604-9 (LNP delivery system); and WO2012/006359 (microparticle delivery systems). LNPs are non-virion liposome particles in which a nucleic acid molecule (e.g. RNA) can be encapsulated. The particles can include some external RNA (e.g. on the surface of the particles), but at least half of the RNA (and ideally all of it) is encapsulated. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

### Cationic Nanoemulsion (CNE) Delivery Systems

In certain embodiments, the nucleic acid-based vaccine comprises a cationic nano-emulsion (CNE) delivery system. CNE delivery systems and methods for their preparation are described in the following reference: WO2012/006380. In a CNE delivery system, the nucleic acid molecule (e.g. RNA) which encodes the antigen is complexed with a particle of a cationic oil-in-water emulsion. Cationic oil-in-water emulsions can be used to deliver negatively charged molecules, such as an RNA molecule to cells. The emulsion particles comprise an oil core and a cationic lipid. The cationic lipid can interact with the negatively charged molecule thereby anchoring the molecule to the emulsion particles. Further details of useful CNEs can be found in the following references: WO2012/006380; WO2013/006834; and WO2013/006837 (the contents of each of which are incorporated herein in their entirety).

Thus, in a nucleic acid-based vaccine of the invention, an RNA molecule encoding a transcript of interest, such as an antigen, may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. In some embodiments, the CNE comprises squalene and a cationic lipid, such as 1,2-dioleoyloxy-3-(trimethylammonio)propane (DOTAP). In some preferred embodiments, the delivery system is a non-viral delivery system, such as CNE, and the nucleic acid-based vaccine comprises a self-replicating RNA (mRNA). This may be particularly effective in eliciting humoral and cellular immune responses. Advantages also include the absence of a limiting anti-vector immune response and a lack of risk of genomic integration.

### SAM Drug Products

SAM Drug Products useful in accordance with the present invention include Drug Products, such as SAM vaccines, that are prepared under process conditions designed to yield a Drug Product that can be administered to a mammal, preferably a human. Thus, SAM Drug Products that can be tested are prepared in accordance with FDA guidelines applicable to the particular Drug Product being tested. The Drug Products are, of course, considered to be safe for administration to humans and are also capable of inducing a protective and/or therapeutic response, such as an immune response, that offers at least some level of protection or therapeutic effect for at least some of the subjects to which it is administered. Thus, the Drug Product will not have any contaminating infectious agents or any agents capable of replication, other than the SAM construct or SAM constructs. In addition, of course, the SAM vaccine will be sterile and free of unacceptable levels of toxic components, which toxic components might otherwise be tolerated in in vitro assays or in vivo animal research studies. Any Drug Product that comprises SAM that encodes an immunogenic or therapeutic polypeptide, such as a viral polypeptide, and a delivery system designed to deliver SAM to mammalian cells so that the SAM can express polypeptide is useful in accordance with the present invention.

Use of Drug Products Tested in Accordance with the Invention

The Drug Products tested in accordance with the present invention are used for inducing an immune or therapeutic response against a pathogen that has a polypeptide corresponding to (but not necessarily identical to) the immunogenic or therapeutic polypeptide encoded by the SAM that is part of the Drug Product. If the Drug Product is a vaccine, the SAM in the vaccine should encode a polypeptide that induces an immune response against a pathogen against which protective immunity is desired.

### Definitions

Unless otherwise indicated by the context, the following definitions apply when used in the present application.

Cationic Nanoemulsion (CNE)—CNEs form nanoparticles from a cationic material, such as a material containing cationic lipids, to which SAM adsorbs.

Drug Product (DP)—A Drug Product is a product that is made according to Good Manufacturing Practices and that is approved for administration to humans, either for clinical trials or commercial sale and use by patients.

Lipid Nanoparticle (LNP)-, LNPs (also sometimes referred to in this application as liposomes) are nanoparticles formed from biodegradable and nontoxic negatively charged lipids (such as phospholipids) that encapsulate the SAM molecules used in the present invention.

Nanoparticles—Nanoparticles are small particles (generally on the order of 1-1,000 nanometers) that are used as part of a delivery system for SAM molecules. Nanoparticles include, without limitation, LNPs, CNEs and polymer-based nanoparticles. Nanoparticles include at least the nanoparticles described by Zhao et al, Nanoparticle vaccines, Vaccine, 32 (2014) 327-337.

Potency—The Potency of SAM drug product means the ability of a full-length SAM comprising 5′ cap and polyadenylated (poly A) tail, formulated with a delivery vehicle, to elicit a biological effect. In particular, the biological effect is the ability of the SAM drug product to elicit the expression of a SAM encoded protein in a cell.

Potency Release Assay—An assay that generates data that predicts and/or confirms potency of a Drug Product before releasing the Drug Product for distribution.

Release Assay—A Release Assay is an assay performed on a Drug Product sample (such as a vaccine sample) from a lot of Drug Product samples prepared under Good Manufacturing Practices (GMP), which Drug Product lot has not yet been released for distribution to distributors, medical professionals or patients. The Release Assay will be performed on one or more Drug Product samples or kits or vaccine samples or kits in a particular lot before the rest of the kits in the lot are released for distribution.

SAM (Self-Amplifying mRNA or SAM replicon) or SAM Molecules—SAM or SAM Molecules useful in accordance with the present invention contain at least mRNA that encodes an immunogenic or therapeutic polypeptide that elicits a preventative or therapeutic immune response or a therapeutic response against infection with a pathogen. The SAM molecule includes elements that allow the mRNA to self-replicate in vitro in a cell culture used in the assay and also to replicate in vivo in an animal and/or human.

SAM RNA Vaccine—A SAM vaccine is a product that comprises a SAM Molecule, formulated with a delivery vehicle, that can elicit an immunological response when administered to a patient.

| Term | Definition |
|---|---|
| APC | Allophycocyanin |
| BHK-21 | Baby Hamster Kidney Cells, ATCC Cell Line CCL-10 |
| BSC | Biosafety Cabinet |
| CNE | Cationic Nanoemulsions |
| CoA | Certificate of Analysis |
| Conc. | Concentration |
| DP | Drug Product |
| DS | Drug Substance |
| Fab | Fragment antigen-binding |
| FBS | Fetal Bovine Serum |
| FITC | Fluorescein Isothiocyanate |
| FSC-A | Forward Scatter Area |
| FSC-H | Forward Scatter Height |
| g | Gravitational Force |
| GFP | Green Fluorescent Protein |
| IVRP | In vitro relative potency |
| LNP | Lipid Nanoparticles |
| mL | milliliter |
| NC | Negative Control |
| PC | Plate Control |
| REF or Ref Std | Reference Standard |
| Rel. Pot. Or RP | Relative Potency |
| RNA | Ribonucleic Acid |
| RT | Room Temperature |
| RV | Reconstituted Vaccines |
| SAM | Self-Amplifying mRNA |
| SMP | SoftMax Pro |
| SSC-A | Side Scatter Area |
| μL | microliter |
| % RSD | Percent Relative Standard Deviation |

### EXAMPLES

### Example 1—Performance of the Assay

Table 1 includes the materials that were used in the assay. All materials are commercially available and each should be treated according to the manufacturer's suggestions.

### Reagents, Chemicals, Solvents and Equipment

NOTE: Unless otherwise specified, equivalent materials may be substituted. All materials must be stored according to manufacturer's directions. Materials should be labeled with date opened, effective expiration date, and initials of analyst.

TABLE 1
Materials

| Materials | Vendor |
|---|---|
| Trypsin EDTA (0.05%) for cell maintenance | Corning |
| Trypsin EDTA (0.25%) for harvest in 96wP | Corning |
| L-Glutamine, 200 mM | Life Technologies |
| Penicillin-Streptomycin Solution, 10,000 U/mL | Corning |
| DNA LoBind Safe-Lock 0.5 mL/1.5 mL Microcentrifuge Tubes | Eppendorf |
| Protein LoBind PCR Clean Tubes 0.5 mL/1.5 mL | Eppendorf |
| BD CytoFix/CytoPerm, 1X | BD Biosciences |
| BD Perm/Wash, 10X | BD Biosciences |
| 96 Well Clear Round Bottom TC-Treated Cell Culture Plate | Falcon |
| BD CS&T Research Beads if using BD FACSVerse flow cytometer | BD Biosciences |
| MACSQuant Calibration Beads | Miltenyi Biotec |
| MACSQuant Running Buffer | Miltenyi Biotec |
| MACSQuant Washing Solution | Miltenyi Biotec |
| MACSQuant Storage Solution | Miltenyi Biotec |
| Anti-dsRNA J2, IgG2a, kappa chain 500 μg | SCICONS |
| Zenon ™ Allophycocyanin mouse IgG Labeling Kit | Invitrogen |
| 1000 mL Nalgene Filter Unit with 0.2 micron PES Membrane | Thermo Fisher |
| | Scientific |
| Aspirating pipet | Corning |
| Filter Pipette Tips (0.1-10 μL, 0.5-20 μL, 2-200 μL, 50-1000 μL, 5 mL) | Eppendorf |
| Extended Length 1000 μL Filter Pipette Tips | Thermo Scientific |
| Serological Pipettes (2 mL, 5 mL, 10 mL, 25 ml, 50 mL) | Fisherbrand |
| VIAFLO Filter Low Retention Pipette Tips (300 μL, 1250 μL) | Integra |
| T225 and T75 culture flasks vented cap | Corning |
| 96-well Deepwell Plate 0.5 mL & 1.0 mL | Eppendorf |
| Conical Centrifuge Tubes, 15 mL | Fisherbrand |
| Conical Centrifuge Tubes, 50 mL | Falcon |
| SAM/LNP Interim Reference Material | GSK |
| SAM/LNP Plate Control Material | GSK |
| BHK-21 Cells | ATCC |
| Dulbecco's Phosphate-Buffered Saline without Calcium and | Corning |
| Magnesium | |
| Trypan Blue Stain 0.4% | Invitrogen |
| Reagent Reservoirs 25 ml | Thermo Scientific |
| 96-Well Collagen I Microplate | Corning |
| DMEM High Glucose Medium | Hyclone |
| Fetal Bovine Serum | Gibco or Seradigm |
| Biotix xTIP4 Pipette Tips 20 μL Racked Sterilized Tips for Costar ® | Biotix |
| Vacuum Aspirator | |
| 8-Channel Adapter for the Costar ® Vacuum Aspirator | Corning |
| Absorbent paper | Texwipe |
| 70% Isopropyl Alcohol | Decon Laboratories, |
| | Inc. |
| Vi-CELL Sample Cups if using Vi-Cell XR Cell Viability Analyzer | Beckman Coulter |
| Cell Counting Chamber Slides if using Cell Countess | Invitrogen |
| Aluminum Foil | VWR |

TABLE 2
Equipment and Software

| Equipment | Vendor |
|---|---|
| Milli-Q Water System Advantage A10 | Millipore |
| Pipette Aid | Integra |
| Calibrated Pipettes (10 μL, 20 μL, 200 μL, 1000 μL, 5000 μL) | Eppendorf |
| Calibrated Multichannel Pipettes, 12-channel 300 μL & 100 μL | Eppendorf |
| Calibrated Electronic Multichannel Pipettes, 12-channel | Integra |
| 1250 μL & 300 μL | |
| 5° C. (±2° C.) Refrigerator | VWR |
| −20° C. (±5° C.) Freezer | VWR |
| −80° C. (±10° C.) Freezer | Revco |
| Biological Safety Cabinet with vacuum aspiration | Baker |
| Vi-Cell XR Cell Viability Analyzer (or Cell Countess) | Beckman Coulter (or |
| | Invitrogen) |
| Miltenyi MACSQuant VYB with MACSQuantify Software | Miltenyi Biotec (or BD |
| (Alternative Flow Cytometer BD FACSVerse with FACSuite | Biosciences) |
| Software) | |
| Phase contrast microscope | Nikon |
| Refrigerated Heraeus Multifuge X3R Centrifuge (w/50 ml tube | Thermo Scientific |
| and multiplate holder) | |
| SoftMax Pro | Molecular Devices |
| FlowJo ™ | FlowJo, LLC |
| MicroSoft ® Excel ® | Microsoft Office |

### Procedure

### Preparations

### Media and Wash Buffer:

The Cell Culture Medium was prepared by combining a sterile bottle of 1000 mL DMVEM with 100 mL FBS, 10 mL L-Glutamine, and 10 mL PenStrep. The Assay Medium was prepared by combining a sterile bottle of 1000 mL DMEM with 50 mL FBS, and 10 mL L-Glutamine. The prepared Cell Culture and Assay media were then filter-sterilized and stored at 5±2° C. for up to 4 weeks. The Wash Buffer was prepared on Day 2, before the experiment, by combining 2 mL FBS to 200 mL DPBS. The Wash Buffer was then filter-sterilized and kept on ice while using. The Wash Buffer was stored at 5±2° C. for the next use, for up to 2 weeks. The volumes of the Cell Culture Medium, Assay Medium, and Wash Buffer each may be scaled accordingly.

Zenon™ APC mouse IgG Labeling Kit:

The handling and storage of the antibody should be done according to the product specification. The antibody should be stored at 2-8° C. Exposure of the antibody to light and freezing-temperatures should be avoided. The working dilution of the primary antibody was prepared freshly on Day 2 and the labeling mix was applied to the cells for staining within 30 min of preparation.

### 1× Perm/Wash Buffer:

The 1× buffer was prepared freshly on Day 2, before the experiment by diluting the 10× BD Perm/Wash buffer with MilliQ water (e.g. combining 9 mL of MilliQ water with 1 mL of 10× Perm/Wash buffer will make 10 mL of IX Wash Buffer). The prepared wash buffer was kept on ice and discarded after using. Volumes may be scaled accordingly.

### Initiation of BHK-21 Cell Culture

NOTE: To maintain sterility of the cells, all work should be conducted in a certified BSC. The CO₂ incubator should be leveled and maintained in good condition to avoid disturbing the even cell seeding pattern in the plates and flasks. To minimize the plate edge effect, a water pan at the bottom level (or an equivalent design) may be used to moisturize the plates and flasks. The centrifuging of cells should be done at 4° C. The Cell Culture Medium should be warmed from 4° C. to room temperature (by placing on bench top). While transporting cells from the storage freezer, it is important to keep the frozen cell vial on dry ice to minimize rapid thawing.

Initiation of a new BHK-21 cell culture was done by first retrieving the cell stock vial from the liquid-nitrogen storage, recording the stock vial, transporting the vial on ice, and placing the vial in a 37° C. water bath without submerging the vial completely. Optimal thawing was visually monitored and, when a small amount of frozen cells was still present, the vial was removed from the water bath. To prevent contamination, the outside of the vial was sprayed with 70% isopropyl alcohol and transferred to a BSC. Excess 70% isopropyl alcohol was removed prior to opening by wiping the outside of the tube. The thawed cells was then gently mixed by pipetting 2-3 times. The cells was introduced to the Cell Culture Medium by transferring the thawed cells into a sterile 50 mL tube and adding 9 mL of warm Cell Culture Medium dropwise to cells with gentle intermittent swirling. The freshly thawed cells were isolated and resuspended in fresh medium by first centrifuging at 300 g for 5 minutes at 4° C., carefully aspirating the supernatant without disrupting the cell pellet, then adding 2 mL of the Cell Culture Medium. The use an extended length 1000 μL pipet tip to gently pipet the cells up and down 10-12 times helped loosen the cell pellet. The addition of an extra 3-5 mL of Cell Culture Medium to the cells diluted the cells helped them to mix evenly.

The cells were counted by adding equal volumes of cells and trypan blue (e.g. 15 μL each), mixing, loading 10 μL to each side of a Cell Countess chamber, and read on the Cell Countess. The cell count (viable, total, dead, % viability) was recorded. Alternate cell counting methods can be utilized (Vi-Cell or hemocytometer) and two counts can be recorded and averaged.

The cells were then reseeded to the final density in pre-warmed Cell Culture Medium according to Table 3.

TABLE 3
Seeding Densities and Cell Culture
Medium Volumes for the Initial Thaw

| 75-85% Confluency at: | T-75 (15 mL) |
|---|---|
| 2 Days | 5.0 × 10⁵, 8.0 × 10⁵, 1.0 × 10⁶ |

NOTE: Initial total cell number may vary. To cover the optimal seeding range, three flasks were initiated at three different densities for two-day seedings if cells were abundant. When cells were ready for passage, the best flask out of these densities with the optimal confluence was used for subculture (at about 75-85% confluent). When the initial vial contained very limited number of cells, one (or two) of the appropriate density was picked and closely monitored for cell confluency to ensure the cells are maintained and subcultured within the optimal confluency range. In addition, the flask size can be reduced to T-25 if the initial vial contains fewer than 5.0×10⁵ cells, and seeding densities will need to be reduced proportionally. The volume of cell suspension needed to seed the flask was determined using the following equation:

$$Inoculation volume ( mL ) = seeding density \div cell count ( viable cells / mL )$$

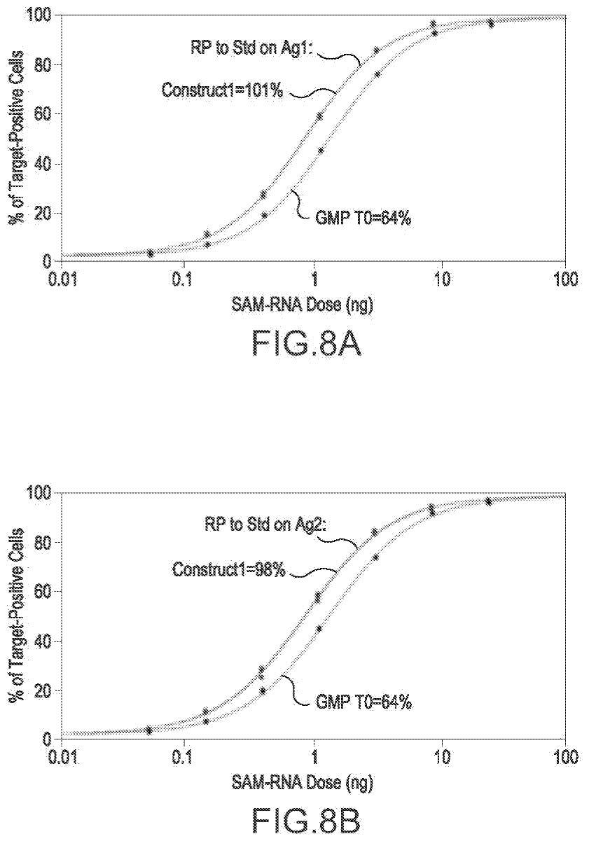

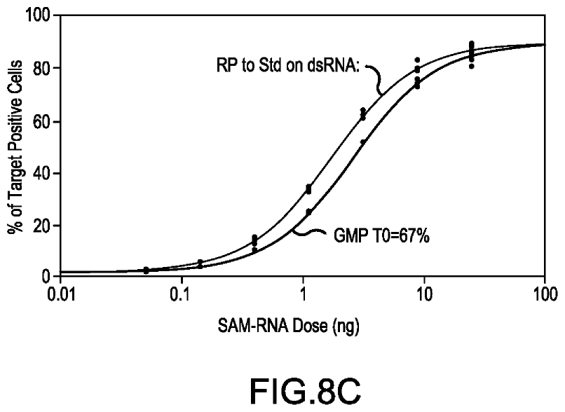

The flask was labeled with a unique identification name, consisting of the cell line, passage number, flask letter (if seeding multiple flasks, each flask was assigned a different letter), seed date, and analyst's initials. “FIG. 8” movements were used to mix the cells evenly and an even seeding pattern was confirmed under the microscope prior to placing the seeded flasks into the incubator. The flasks were incubated at 37±1° C., 5±1% CO₂ and elevated humidity. The Cell Culture Medium was changed 24-28 hours after seeding, then every 2 days until the cells were about 70-85% confluent and ready for subpassaging. Usually, the cells were ready for sub-passaging approximately two days later.

### Maintenance of Existing BHK-21 Cell Cultures

As with initiation of BHK-21 Cell Cultures, the NOTE above also applies to the maintenance of the cell cultures.

The BHK-21 cells were preferred to be sub-passaged on a Monday, Wednesday, and Friday schedule for up to 25 additional passages, at which time the cells were discarded. Friday subpassaging were done in the late afternoon, considering the longer weekend break than the rest of the schedule.

NOTE: To prevent contamination, the outside of the vials or material can be sprayed with 70% isopropyl alcohol before transferring them into the BSC.

Trypsin/EDTA (0.05%) was used to dislodge cells from the flask in the subpassaging procedure. Only warm Trypsin/EDTA should be added to the cells, and Trypsin/EDTA should be removed from −20° C. storage and placed in a 37° C. water bath until equilibrated. Cells should be centrifuged at 4° C. and the centrifuge should be equilibrated to 4° C. prior to use with the cells.

Prior to subculturing, BHK-21 cells were inspected macroscopically and microscopically for morphology and contamination and the details recorded (passage #, date plated, density, culture flask size, and operator). The cell cultures from the Initiation-of-BHK-21-Cell-Culture procedure described above that achieved a seeding density of 70-85% confluency were selected for further subculture.

Before transferring the cells to a new flask, the media was first removed by carefully aspirating using a sterile pipette. To avoid cell loss, the pipette was positioned away from the cell surface in the T225 culture flask. The attached cells were then washed by adding 25 mL room-temperature DPBS, gently swirling the flask, and aspirating the DPBS.

TABLE 4
Reagent Volumes

| Flask Size: | PBS Wash | Trypsin/EDTA |
|---|---|---|
| T-75 | 10 mL | 3 mL |
| T-225 | 25 mL | 5 mL |

The attached cells were then dislodged and transferred to a new flask by first adding 5 mL of warm Trypsin/EDTA per T225 culture flask to dislodge them from the old flask. The flask was rocked at 37° C. for 5.0±0.5 minutes to help distribute the Trypsin throughout the cells in the flask. To ensure that the cells were fully dislodged from the flask, they were checked under a microscope.

A new culture was started with the trypsinized cells by adding an equal volume of Cell Culture Medium to the cells, pipetting up and down with a 10 mL serological pipet 3-5 times, and transferring to a 50 mL tube. Cells were then collected by centrifuging at 300 g for 5 minutes at 4° C. and carefully aspirating the supernatant without disrupting the cell pellet. The cells were then given fresh Cell Culture Medium by gently resuspending the cell pellet in 2 mL of Cell Culture Medium. An extended length 1000 μL pipet tip was used to gently pipet the cells up and down 10-12 times; thus ensuring thorough mixing of cells in the fresh Cell Culture Medium. An additional 8 mL of Cell Culture Medium was added to dilute the cells and mix them evenly.

Cell number was analyzed using the procedure described above in the Initiation of BHK-21 Cell Culture. Viability must be greater or equal to 85% and confluency less than 90% to passage the cells. The reseeding of the cells can be completed by adding pre-warmed cell culture medium to a final density according to Table 5. “FIG. 8” movements were performed to mix the cells evenly. The flasks of cells were returned to the incubator to resettle and grow.

TABLE 5
Suggested Seeding Densities and Cell Culture Medium Volumes

| 75-85% Confluency at: | T-225 (40 mL) |
|---|---|
| 1 Day | 5.0 × 10⁶ |
| 2 Days | 1.0 × 10⁶, 1.4 × 10⁶ |
| 3 Days | 5.0 × 10⁵, 6.0 × 10⁵, 7.5 × 10⁵ |

NOTE: To cover the optimal seeding range, two and three-day seedings were performed with at least 2 and 3 flasks at different densities, respectively (see suggested densities in Table 5). When cells were ready for passage, the best flask out of these densities with the optimal confluency was used for subculture (at about 70-85% confluent). Considering the variation of cell doubling time, cell seeding density can be flexible and adjusted when needed.

DAY 1—Delivery of SAM mRNA by LNP into BHK-21 Cells

The NOTE previously emphasized with Initiation of BHK-21 Cell Culture and Maintenance of Existing BHK-21 Cell Cultures also applies here. The Trypsin/EDTA and Assay Medium to be used on the cells were pre-warmed to 37° C. (using a water bath) and room temperature (by placing on the bench top), respectively, until equilibrated. All necessary materials for Day 1 treatment were retrieved except for the SAM/LNP. All needed tubes, reagent reservoirs, and plates were labeled according to the plan for the harvest. The centrifuge temperature was set to 4° C. prior to use with cells.

Preparation for the harvest of BHK-21 Cells for Non-Viral Delivery of SAM began with the inspection of the cells macroscopically and microscopically for morphology and contamination. BHK-21 cell details (passage #, date plated, density, culture flask size, and operator) were recorded. To ensure they are in optimal growth phase for the assay, BHK-21 cells should have been maintained and harvested as described above in the Initiation of BHK-21 Cell Culture and Maintenance of Existing BHK-21 Cell Cultures sections.

Cells should be trypsinized, isolated, given fresh medium, and re-plated as described above. Viability should be greater or equal to 85% to use for the assay. Readings need to be similar (as close as possible) between duplicates (difference ≤10% preferred, redo if >15%),

Cells were diluted for the Non-Viral delivery of SAM by first calculating the volume of viable cells. The volume of viable cells was calculated with some extra (e.g. 10 mL) that corresponds to the number of cells needed for seeding the appropriate number of 96-well plates (8.0E+04˜1.0E+05 cells per well, 150 μL per well).

Notes: 1. Density was determined by in-house stock of BHK-21 cells counted with Cell Countess. Considering the variation of cell count results among different counting methods/labs/analysts, and variation of cell double time among different passages and lots of BHK cells, seeding number can be adjusted slightly when necessary to avoid either over-confluent culture or insufficient events at Day 2. 2. To be more QC friendly, the range of seeding density calculated in the dilution step above and overnight SAM/LNP incubation time length (after cells are plated) were widened. The lower seeding density (8.0E+04) was paired with a longer overnight incubation (e.g. 19 h) while a higher density (e.g. 1.0E+05) was paired with a shorter overnight incubation (e.g. 16 h) to ensure a good number of cells without getting the cell layers lifted up due to over-confluency at harvest.

A sample calculation of plate number using the samples listed above (every plate for testing up to 3 samples duplicated wells) is:

$$Total volume of diluted cells containing the extra 10 mL is : ( \# of plates ) \times 15 + 10 = 25 mL .$$

If the Average Viable/mL=1.0E+06 viable cells/mL, the volume of cells needed:

$$[ ( ( \# of plates ) \times 15 + 10 ) \times ( 1. E + 05 ) / 0.15 ] / [ Average Viable / mL ]$$

For 1 plate: [((1 plate)×15+10)×(1.0E+05)/0.15]/[1.0E+06 viable cells/mL=16.7 mL

All needed tubes, reagent reservoirs, and plates were labeled according to the plan for the harvest. The dilutions listed on Table 6 should be included for each of standard, plate control(s), and test sample(s). The SAM/LNP Interim REF, plate control lot, and Test Samples if there is any, were removed from −80° C. and kept on a 2-8° C. bead bath (˜0.5 hr before the treatment dilution preparation starts). The caps of frozen vials were tightly closed during storage to avoid changes of concentration, stability, and formulation with LNP, which may impact the accuracy of the assay.

TABLE 6
Example of Dilutions for Each of Standard,
Plate Control(s), and Test Sample(s).

| Samples | n = |
|---|---|
| Interim REF/PCs/Samples 7 (25 ng SAM/LNP RV) (R/PC/S -7) | 2 |
| Interim REF/PCs/Samples 6 (8.929 ng SAM/LNP RV) (R/PC/S -6) | 2 |
| Interim REF/PCs/Samples 5 (3.189 ng SAM/LNP RV) (R/PC/S -5) | 2 |
| Interim REF/PCs/Samples 4 (1.139 ng SAM/LNP RV) (R/PC/S -4) | 2 |
| Interim REF/PCs/Samples 3 (0.407 ng SAM/LNP RV) (R/PC/S -3) | 2 |
| Interim REF/PCs/Samples 2 (0.145 ng SAM/LNP RV) (R/PC/S -2) | 2 |
| Interim REF/PCs/Samples 1 (0.052 ng SAM/LNP RV) (R/PC/S -1) | 2 |
| Total Wells per Treatment (REF or PC or S) | 14 |

The volume of viable cells that corresponds to the number of cells needed for treatments seeding the 96-well plates (1.0E+05 cells per well) were transferred to a sterile bottle or tube. The right volume of Assay Medium was added to bring to a final density of (6.67E+05 cells/mL). The samples were kept on ice. An example calculation is: For 96w Plates: (1 plate*15 ml/plate+10-16.7 mL=8.3 mL. The diluted cell mix was stored on the 2-8° C. bead bath when the treatment preparation could not be started right away.

The delivery of SAM/RNA by LNP into BHK-21 Cells procedure began with retrieval of SAM/LNP Interim REF, PC, and Test Samples if there were any, from −80° C. storage and kept on the 2-8° C. bead bath. DNA LoBind Eppendorf tubes, Comical tubes, 96-well Deepwell plate, and reagent reservoirs needed for the treatment were labeled. The appropriate volume of Assay Medium were added to each labeled tube based on the calculation on Table 7. The appropriate volume of Assay Medium was also added into corresponding wells of the Deepwell plate. An example of 2.8× serial dilution in a 96-well Deepwell plate is as follows: Load 288 μL of Assay Medium to rows B through H of the plate; for up to three samples, add two more samples S2 and S3 into columns 4, 5, 9, and 10 with sample preparation strategy (load 448 μL or S2-7 and S3-7 instead of Assay Medium in wells A4/9 and A5/10, respectively). For assay development and qualification, the same Ref Std lot material was used for PC to better monitor the assay performance.

TABLE 7
An Example of SAM-RNA Dilutions.
Independent duplicates for Interim REF/PC/Sample(s) were prepared.

| | Final | Final | LNP Used for Dilution | | | Assay | Total |
|---|---|---|---|---|---|---|---|
| | Conc. | Dose | | Conc. | LNP | Medium | Volume |
| Sample | (μg/mL) | (ng/well) | Material | (μg/mL) | (μL) | (μL) | (μL) |
| Interim | 0.5 | 25 | Ref Std | X | 10.0 | 10*X/0.5-10 | 10*X/0.5 |
| REF(R7A/B) | | | | | | | |
| PC (PC7A/B) | 0.5 | 25 | Plate | Y | 10.0 | 10*Y/0.5-10 | 10*Y/0.5 |
| | | | Control* | | | | |
| Sample (S-7A/B) | 0.5 | 25 | Sample | Z | 10.0 | 10*Z/0.5-10 | 10*Z/0.5 |

Dilutions of REF/PC/Samples were prepared in a tight time frame without disturbance. The dilution starting time point (T1) and the time point when cells were loaded onto the diluted treatment in 96-well Collagen plate (T2) were recorded. It is important to ensure that the waiting time of diluted LNP in medium before the treatment starts (T2−T1) is well controlled without prolonged delay, and is comparable among Interim REF, PC(s), and samples. For each individual 96-well Collagen Plate, the treatment preparation and cell seeding were finished individually (from this step to the incubation of the plates in 37° C. after delivery of SAM RNA by LNP (see below)) before moving on to the next plate. Also, it is important to finish the treatment for all plates within a reasonable time to limit the variation of waiting time for the diluted cells before seeding.

The appropriate volume of stock Interim REF/PC/Sample material was added into the corresponding tubes (R7/PC7/S1-7) quickly and T1 recorded. Serial dilutions of SAM/LNP were performed by transferring 448 μL of R7/PC7/S1-7 prepared to the corresponding wells of the labeled Deepwell plate. A multichannel pipette was used to transfer 160 μL of R7/PC7/S7 into the next dilution R6/PC6/S6. The 1:2.8 dilution was fully mixed by gently pipetting up and down 5 times. The 1:2.8 serial dilution was repeated until R1/PC1/S1. For the analyst's convenience, a serial dilution with a 12-channel pipette can be used to do overall serial dilutions for all 12 columns. A pipette was used to quickly load 50 μL/well of R7-R1/PC7-PC1/S1-7-S1-1/medium.

An electronic 1250 μL, 12-channel pipette was set with 25 μL predispense and 25 μL postdispense. The diluted BHK cells were quickly resuspended/mixed, then transferred into a sterile reservoir. The 1250 μL electronic multichannel pipette was used to mix and carefully load 150 μL/well of cell suspension onto the layer of treatment in the 96w cell culture plate from row H to row A. The plate was gently shaken as to not splash the medium onto the lid (optional). The cell pattern was checked under the microscope for even distribution and suspension in the 96w plate. The plate was then incubated in 37° C.; 5% CO₂ incubator for 18±2 hr. The entire procedure was repeated for the remaining plates if there were any additional. One full plate can test up to 3 samples.

### Day 2—Immunostaining and Flow Analysis of SAM/LNP-Transfected BHK-21 Cells

Day 2 Preparation was set up as described above: 0.25% Trypsin/EDTA was warmed to 37° C. and the Cell Culture Medium to room temperature. The Wash Buffer, BD CytoFix/CytoPerm (1×), and BD Perm/Wash (10×) were kept in on ice-cold bead bath or ice bucket during use. 1× Perm/Wash buffer was prepared freshly as described in the Preparations section above.

The lyophilized anti-dsRNA J2 antibody was reconstituted with 500 μl sterile distilled water per 500 μg antibody following the vendor's instructions, then aliquoted into Protein-Low bind Eppendorf tubes and stored at −70° C. freezer. During the waiting time of the upcoming centrifuge step (see below), aliquoted mouse Anti-dsRNA J2 antibody can be removed from −70° C. and Zenon™ APC mouse IgG2a Labeling Kit from 4° C. and placed in an ice cold bead bath or ice bucket. Exposure of Zenon™ APC mouse IgG2a Labeling Kit to light should be avoided. The flow cytometer was turned on 30 minutes before reading to allow time for the instrument to warm up and the centrifuge was set to 4° C.

Day 2 harvest of RG.Co.2-mRNA-Transfected BHK-21 Cells began with the use of a sterile multi-Channel Adapter and Costar® Vacuum Aspirator to aspirate the medium from the corner of wells in the 96-well culture plates that contained the treated cells. In order to avoid disturbance of BHK cell layer, the media was aspirated from the surface of the medium with mid-high vacuum speed instead of maximum speed. The cells were harvested one plate at a time in the same order of treatment that was completed on the previous day, at 16 ˜ 20 hr after incubation in CO₂ incubator.

A 1250 μL electronic 12-channel pipette with 25 μL predispense and 25 μL postdispense was used to add 250 μL DPBS without Calcium and Magnesium to each well of the 96-well culture dish by electronic pipette gently from row H to row A. The postdispense DPBS was discarded into a liquid waste container, the pipette tips changed in the middle (after row H/G/F/E), and the media gently swirled and aspirated. 125 μL pre-warmed Trypsin/EDTA was added to each well on the 96-well culture dish quickly with the electronic 12-channel pipette from rows H to A and distributed by gently tilting the plate. The 96-well plate was then placed in the 37° C. incubator for 5.0±0.5 minutes. The multichannel pipette was used to gently pipet up and down a few times toward the corner and center of wells to help cell detach into a uniform cell suspension. The cells were checked under the microscope quickly to ensure that they were dislodged. 125 μL Cell Culture Medium was then added to the cells right away (within 10 min from Trypsin/EDTA loading time), and gently pipetted up and down 3 times to thoroughly mix the cells.

The cell suspension was transferred into a U-bottom plate. A balance plate containing the same volume of liquid (MilliQ water or buffer) was prepared for the harvested plate, and the liquid volume of the balance plate was adjusted in each centrifuge step when there was a volume change in the harvested plate.

In order to expose cells to CytoFix/CytoPerm, the cells were first washed by centrifuging the plates at 300 g for 5 minutes at 4° C. The supernatant was discarded into a waste container. To remove excess liquid, the plate was placed upside down on absorbent paper to blot the remaining liquid. A P300 multichannel pipet was then used to add 200 μL wash buffer to each well. To resuspend the cell pellet, the mixture was pipetted up and down ˜5 times. This wash step was repeated by again centrifuging the cells and removing the supernatant as just described.

Using a P300 multichannel pipet, 100 μL of CytoFix/CytoPerm buffer was added to each cell pellet and mixed by pipetting up and down 5-10 times to ensure that each cell pellet was fully dispersed into a single cell suspension. The plates were cooled in a 4° C. refrigerator for 15-20 minutes with the lid on. An additional 150 μL of 1× Perm/Wash buffer was then added to each well and pipetted up and down 3 times, and the cells were centrifuged at 300 g for 5 minutes and 4° C.

During the last centrifuge step with the 1× Perm/Wash buffer, the antibody mix was prepared (see antibody preparation notes on Day 2 described above). The supernatant was again removed as described in the wash steps. The appropriate amount of PFA needed was loaded into the reservoir. All PFA-containing waste including reservoir and PFA-absorbed tissue were collected into a tightly sealed PFA-waste-specific container. The cell pellets were resuspended in 200 μL 1× Perm/Wash buffer by pipetting up and down 3-5 times using P300 multichannel pipet.

The cells were centrifuged at 300 g for 5 minutes at 4° C. and the supernatant removed as described in the wash steps above.

A mix of anti-dsRNA J2 antibody/Zenon™ APC mouse IgG2a labeling kit was diluted in 1× Perm/Wash buffer at 1:1000 v/v for anti-dsRNA Ab and 1:200 v/v for Zenon™ APC mouse IgG2a labeling kit for 100 μL/sample were prepared as follows:
1. PER SAMPLE to stain, V1 μL anti-dsRNA J2 Ab was mixed with the indicated volume (V2A) of component A and incubated for 5 minutes at room temp.
2. Excess Fab was blocked by adding indicated volume (V2B) of Component B PER SAMPLE and incubated for 5 minutes at RT.
3. The appropriate volume of Perm/Wash buffer PER SAMPLE was added to dilute the antibody mix to working concentration.
4. The diluted antibody mix was applied to the cells within 30 minutes of preparation.

TABLE 8
Example Calculation using the samples listed above.

| | | Zenon ™ IgG antibody kit | | | |
|---|---|---|---|---|---|
| Sample | Primary | component | component | Perm/Wash | Total Volume |
| Number | Ab (μL) | A (μL) | B (μL) | (μL) | (μL) |
| X | V1 = Y/1000 | V2A = Y/200 | V2B = Y/200 | =Y − V1 − V2A − V2B | Y = X*100 + 1000 |

$$\begin{matrix}( 80 wells in a full plate for 3 samples ) \times ( 100 μL / sample ) = 8 mL \to 9 mL with 1 mL extra antibody solution \\ ( 9 mL ) / 1000 = 9 μL Anti ‐ dsRNA J 2 primary antibody \\ ( 9 mL ) / 200 = 45 μL component A in {Zenon}^{T M} APC mouse IgG antibody kit \\ ( 9 mL ) / 200 = 45 μL component B in {Zenon}^{T M} APC mouse IgG antibody kit \\ ( 9 mL - 9 μL - 45 μL - 45 μL ) = 8901 μL Perm / Wash buffer needed\end{matrix}$$

100 μL of the diluted antibody mix was added to each well with the P100 or P300 multichannel pipet, pipetted up and down 3-5 times to resuspend the cell pellet. 100 μL of Perm/Wash buffer was added into each of the rest of the empty wells of the plate. In order to avoid exposure to light, the plate was wrapped with aluminum foil. The wrapped plate was incubated for 45+5 minutes in a 4° C. refrigerator.

After incubation, an additional 150 μL of Perm/Wash buffer was added to each well and the cells were very gently pipetted up and down 3 times. The plates were centrifuged at 300 g for 5 minutes at 4° C. The supernatant was removed by flicking into a waste container and excess liquid was blotted away by placing the plate upside down on absorbent paper. 200 μL Perm/Wash buffer was added and pipetted up and down 3-5 times to resuspend the cell pellet. Pelleting of cells and washing of cells are repeated and performed as previously described. 70-90 μL Wash Buffer was added to each well of the plate and pipetted up and down 3-5 times to resuspend the cell pellet.

Note: Considering the variation of cell loss during the staining, seeding density, and cell counting (especially when performed by a newly trained analyst), if the final cell pellet is overall smaller than normal, use a small volume (e.g. 70 μL instead of 90 μL) to resuspend the cell pellet for flow cytometer reading. Wrap the plate with aluminum foil to avoid light and store it in 4° C. refrigerator before the flow cytometer is ready to read.

Detection with Flow Cytometry was performed using a Miltenyi MACSQuant VYB or FACSVerse or equivalent flow cytometer. The flow cytometer waste was emptied and sheath fluid container was ensured to be full. The user manual was followed in turning on and warming up the lasers. For VYB, the flow cytometer was switched to acquisition mode and the system was primed. It was daily cleaned for VYB: Run a Clean program was used by right-clicking the Rinse button and selecting Clean. The on-screen instructions were followed: a. 0.5 mL of a 1% hypochlorite solution was added into a 5 mL tube; b. Continue was clicked; c. The cleaning process took approximately 10 minutes. Run a Flush program was performed by right-clicking the Rinse button and selecting Flush. The process took about 15˜18 minutes.

The MACSQuant software manual was followed to run automatic PMT beads calibration for VYB. Briefly: the Single tube rack was ensured to be correctly attached; the Barcode button on the toolbar was clicked to activate the 2D code reader; the 2D barcode printed on the vial label of the MACSQuant Calibration Beads was scanned and the dialog Box instructions followed; the MACSQuant Calibration Beads were thoroughly vortexed or shaken to break up any aggregates; one drop was dispensed into an empty tube and placed in the Single tube rack; “OK” was pressed to start the calibration. Successful calibration for each channel was indicated by a green check mark. When the process was successfully completed, the MACSQuant Instrument Status bar reported Acquisition Mode: Calibration OK.

The software manual was followed to set up the experiment: under Experiment tab, the plate type was selected as “Chill 96 Rack”, the wells that need to be tested were selected. The order was read from left to right, Low flow rate, Mix Gentle, mode Fast, uptake volume 25 μL, input the rough μL of sample total volume/well. The Y3 channel was renamed to APC or dsRNA (optional); previously saved instrument settings and analysis template for dsRNA staining (e.g. Y3 560 v, trigger 30, etc.) were loaded and applied.

The experiment settings were reviewed and confirmed under View >Experiment table, Chill 96 Rack the plate without lid with the right direction was loaded. “Run” was clicked. At the end of the run, all files were exported into FCS files. All experiment related data files were copied from the equipment build-in computer into specific for future remote analysis by FlowJo or other equivalent software.

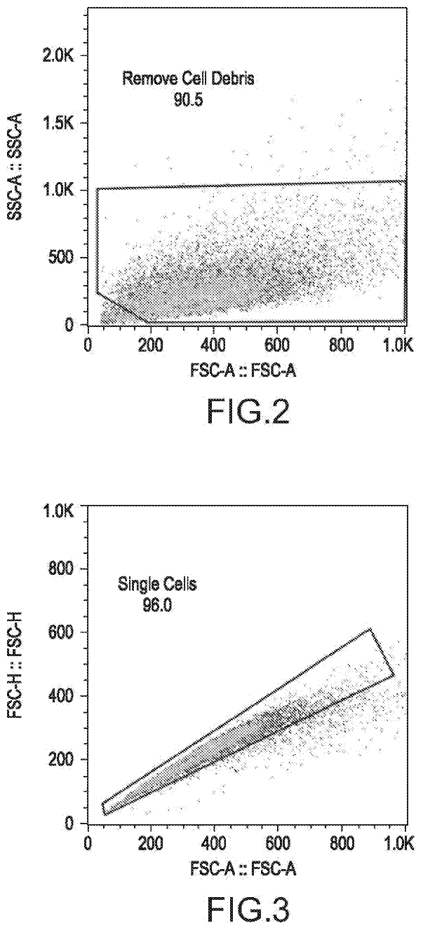

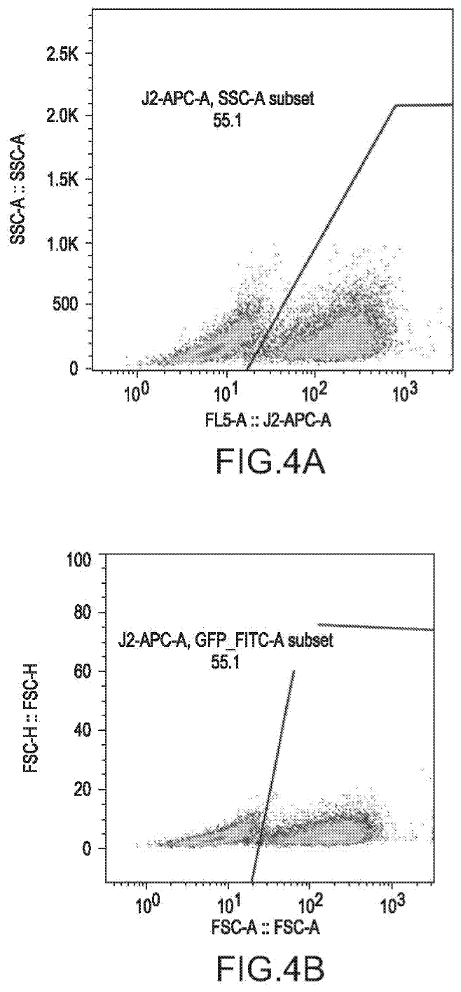

The analysis of sample data was done by loading the data into FlowJo or equivalent software and ensuring proper gate placement. Each sample was checked and verified that the first gate (P1) excludes cellular debris (as below). Adjustments were made as necessary. FIG. 2 is an example of a First Gate (P1): SSC-A vs. FSC-A. Each sample was checked and verified that the second gate (P2) (FSC-H vs. FSC-A; encompasses only single cells and excludes doublets (as below). Adjustments were made as necessary. FIG. 3 is an example of a Second Gate (P2): FSC-H vs. FSC-A. Each sample was checked and verified that the boundary of the third gate (APC-A) (SSC-A vs. APC-A or GFP-A vs. APC-A) rests in between the negative population (cells to the left) and the positive population (cells to the right). Adjustments were made as necessary. FIGS. 4A and 4B illustrate the boundary of a third gate (P3), which plots SSC-A vs APC-A (FIG. 4A) or GFP-A vs. APC-A (FIG. 4B). The % APC positive cells of all were exported into an excel data sheet and converted into 96-well plate format based on their well ID. An excel sheet template was designed to convert the raw exported excel data into a 96-well plate format table for SMIP analysis in the next step.

The Post-Analysis of Standards and Sample Data was performed using SoftmaxPro. “File” was clicked and “Open” was selected. The location where the dsRNA Assay SMIP Analysis Template was located and “Open” was clicked. With the data organized corresponding to the plate, the data was copied, the “A1” well of Softmax plate template was selected and right clicked, and “Paste Data” selected. The dsRNA Assay SMIP Analysis Template plotted the dose response of SAM/LNP standards, plate control, and samples; then, fitted them to a 4-parameter curve, as well as evaluated the Sample data against this curve to determine the Sample's Relative Potency.

### Reporting of Result

The final reportable result of SAM/LNP dsRNA relative potency was rounded to a whole integer following the departmental procedures. The IVRP was calculated by global fit of the sample (or Plate Control)'s curve to the standard curve.

### System and Sample Suitability

The following acceptance criteria were proposed and applied independently to each assay plate (Table 9). The proposed suitabilities were confirmed or revised after the assay qualification based on the qualification results. All confirmed acceptance criteria for the Reference Standard and Standard Curve must be met for each assay for the sample results to be considered further.

TABLE 9
Proposed System and Sample Suitability Criteria

| | | Criteria |
|---|---|---|
| System Suitability Parameter | | |
| Interim | % RSD of Duplicate Values | ≤30% |
| REF | Reference Standard fit to 4PL Regression; (R2) | ≥0.97 |
| Plate | % RSD of Duplicate Values | ≤30% |
| Control | Potency Range (%) | 70% ≤ x CoA ≤ 130% |
| | Slope (B) Ratio to Reference Standard Curve | 0.75 ≤ B Ratio ≤ 1.33 |
| Sample Suitability Parameter | | |
| Test | Slope (B) Ratio to Reference Standard Curve | 0.75 ≤ B Ratio ≤ 1.33 |
| Samples | | |

### Example 2—Demonstration of Correlation Between Total dsRNA and Antigen Expression for Various SAM/LNP Drug Products as Measure of Vaccine Potency

A number of experiments were performed to determine the efficiency of various different SAM/LNP formulations. The following batches of SAM/LNP vaccines were tested in this Example.

| Batch No. | Construct Description |
|---|---|
| 1 | HSVLNP GMP T0 |
| 2 | HSVLNP Tox T0 |
| 3 | HSVLNP Tox T 2 W 25 C. |
| 4 | CMVLNP N68721-76 Sample1 |
| 5 | CMVLNP N68721-76 Sample2 |
| 6 | CMVLNP N68721-76 Sample3 |
| 7 | CMVLNP N68721-76 Sample4 |
| 8 | CMVLNP N68721-76 Sample5 |
| 9 | CMVLNP N68721-65 Sample1 |
| 10 | CMVLNP N68721-65 Sample2 |
| 11 | CMVLNP N68721-65 Sample3 |
| 12 | RabLNP UnMethylated (to FL) |
| 13 | RabLNP UnCapped (to FL) |
| 14 | RabLNP No Tail (to FL) |

Each batch number represent a different batch of SAM formulated in LNP. There are 3 batches of SAM targeting the HSV virus (batches 1-3), 8 targeting the CMV virus (Batches 4-11), and 3 targeting *Rab*ies (Batches 12-14). The 3 HSV batches each have a slightly different purity of target full length nucleic acid sequence to encode the same antigen glycoprotein gE and gI. Likewise, the 8 CMV batches differ at the purity of target full length nucleic acid level, but all encode a common antigen glycoprotein gB. Same for the 3 rabies batches that all encode common antigen glycoproteins.

The SAM/LNP formulation consisted of approximately 60 μg of SAM and about 0.9˜3.6 mg of lipids (RV39, PEG, Cholesterol, and DSPC) per ml of an aqueous buffer containing Tris, NaCl, and Sucrose. Most of the mRNA was encapsulated in liposomes by rapidly mixing lipids and lipophilic components in an organic phase with the hydrophilic SAM dissolved in an aqueous phase using Nanoassemblr Microfluidic Chip. The organic phase and mixing buffer are then removed to get SAM LNP drug product. Details on how to encapsulate the mRNA in liposomes are set forth in the following publications. Anderluzzi et al, Investigating the Impact of Delivery System Design on the Efficacy of Self-Amplifying RNA Vaccines, Vaccines (Basel), Vol. 8, No. 2 (June 2020).

Total dsRNA levels were measured using flow cytometry by the techniques described above. Levels of antigen expression were measured by a monoclonal antibody to detect the antigen of interest.

### Example 2-1—Correlation Between Antigen Expression and dsRNA—CMV Ag1/LNP

### Sample Test Example 1

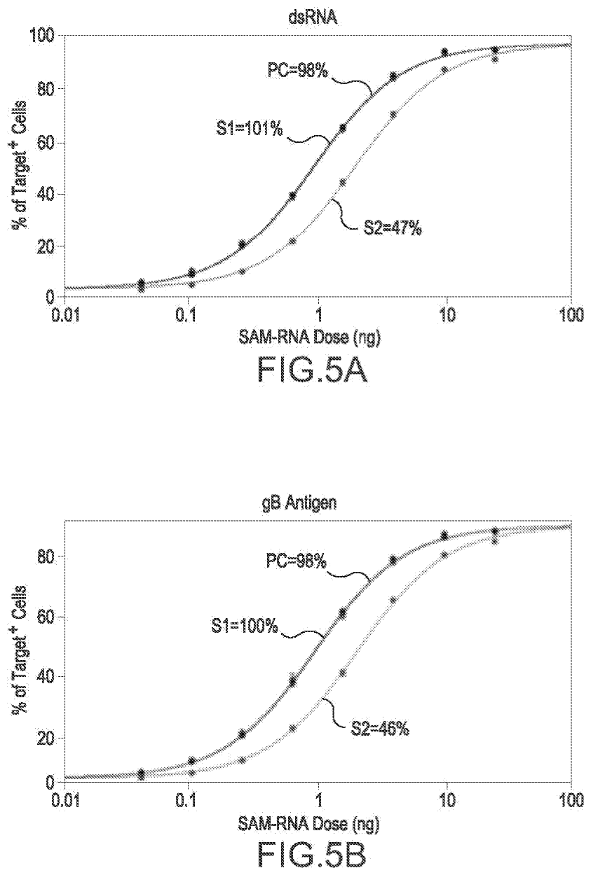

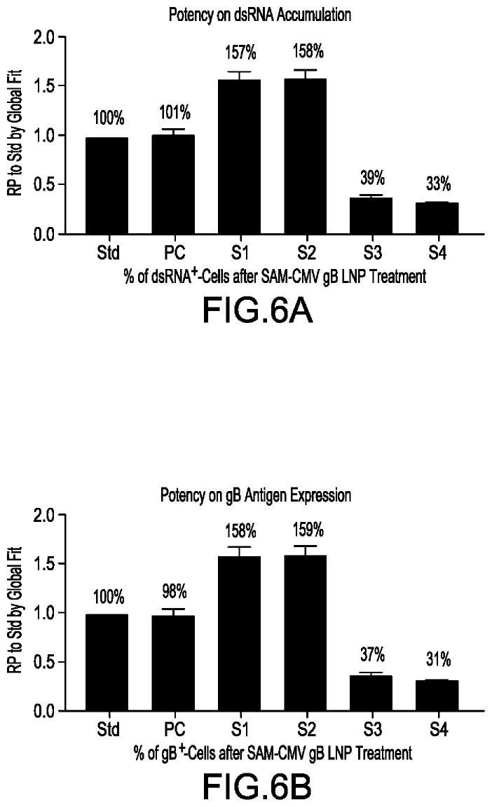

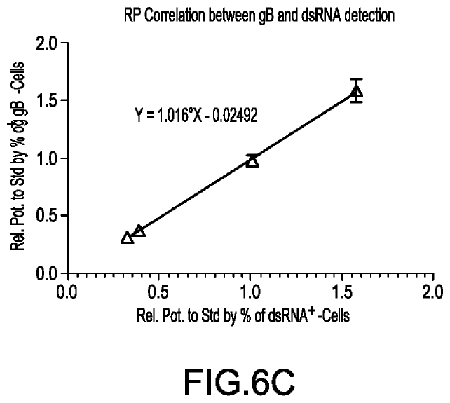

FIG. 5A is a graph showing the amount of dsRNA in target cells based on the amount of target positive cells at various SAM doses for three different SAM constructs encoding CMV-Ag1 in a lipid nanoparticle delivery system.

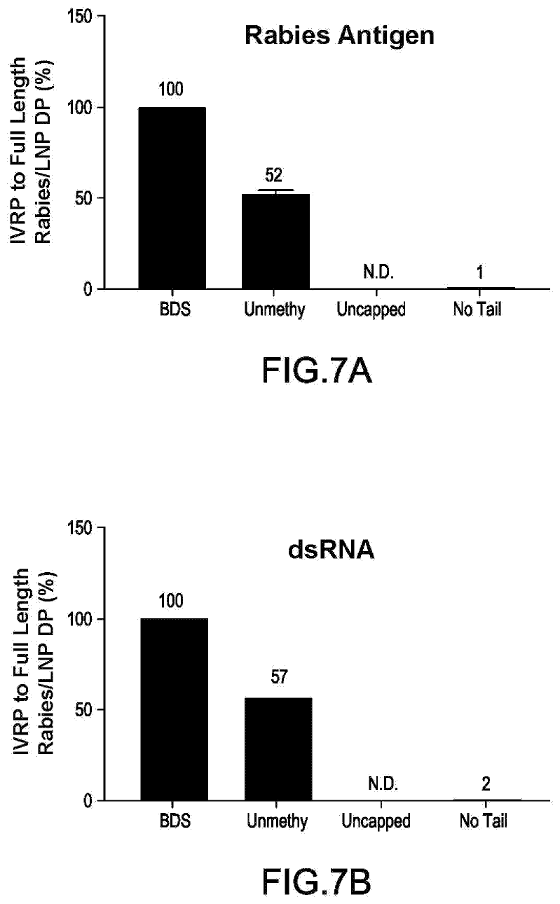

FIG. 5B is a graph showing the amount of gB antigen in the same target cells used to generate the data in FIG. 7A based on the amount of target positive cells at various SAM doses for three different SAM constructs encoding CMV-Ag1 in a lipid nanoparticle delivery system.

The samples measured in FIGS. 5A and 5B have correlated relative potency values (calculated in accordance with the description in Example 1) from antigen expression and dsRNA assays as reported in the Table below (where the IVRP (in vitro relative potency) are recorded):

| CMV CMV_Ag1LNP | | |
|---|---|---|
| IVRP(%) to Std | dsRNA | CMV_Ag1 Antigen |
| Sample1 | 98 | 98 |
| Sample2 | 101 | 100 |
| Sample3 | 47 | 46 |

### Sample Test Example 2

This Example describes another experiment, similar to Sample Test Example 1, that demonstrates that that testing with the dsRNA antibody produces very similar results to testing carried out using an antibody that binds the expressed protein antigen.

FIG. 8A is a graph showing potency on dsRNA accumulation by comparing relative potency (RP) to standard by Global Fit to the % of dsRNA⁺ cells after SAM-CMV gB LNP treatment.

FIG. 8B is a graph showing potency on gB antigen expression by comparing RP to standard by Global Fit to the % of gB⁺ cells after SAM-CMV gBLNP treatment.

FIG. 8C is a graph showing RP correlation between gB and dsRNA detection by comparing relative potency to standard by % of gB⁺ cells to relative potency to standard by % of dsRNA⁺ cells.

The samples measured to create FIGS. 8A, 8B and 8C have relative potency values from antigen expression and dsRNA assays as reported in the Table below (where the IVRP (in vitro relative potency) are recorded):

| CMV CMV_Ag1LNP | | |
|---|---|---|
| IVRP(%) to Std | dsRNA | CMV_Ag1 Antigen |
| Sample1 | 101 | 98 |
| Sample2 | 157 | 158 |
| Sample3 | 158 | 159 |
| Sample4 | 39 | 37 |
| Sample5 | 33 | 31 |

The above data demonstrate that samples have correlated relative potency values from antigen expression and dsRNA assays.

### Example 2-2—Correlation Between Antigen Expression and dsRNA—Rabies/LNP

This example investigates the impact of SAM structural integrity on SAM-Rabies/LNP potency. The results of different rabies constructs (transcripts of interest) are illustrated in the following Table below (where the IVRP (in vitro relative potency) are recorded) and are illustrated in FIGS. 7A and 7B.

| Rabies-LNP IVRP(%) to FL | dsRNA | Rabies |
|---|---|---|
| Full Length Product | 100 | 100 |
| UnMethylated | 57 | 52 |
| Uncapped | N.D. | N.D. |
| No tail | 2 | 1 |

The above results show that a comparable impact of methylation, cap, and tail on SAM-Rabies Potency was observed in (1) antigen expression with fixed cells by flow cytometry (characterization assay) and (2) dsRNA accumulation with fixed cells by flow cytometry (release assay). As expected, the full-length RNA product delivered the highest relative potency in comparison to the chemically and structurally incomplete RNA (unmethylated, uncapped, or without-the-poly-A tail) products. Nonetheless, regardless of the type of RNA construct delivered to the cells, the expression of dsRNA and Rabies antigen (via flow cytometry measurement) were surprisingly similar, emphasizing the correlation between dsRNA and specific-antigen expression after SAM drug product delivery.

### Example 2-3—Correlation Between Antigen Expression and dsRNA—HSV/LNP

### Sample Test Example 1

FIGS. 8A, 8B and 8C are graphs showing the correlation between antigen expression (% antigen expressing cells) and dsRNA (FIG. 8C) for HSV/LNP, wherein the antigen measured in FIG. 8A is gE antigen and the antigen measured in FIG. 8B is gI antigen. The graphs show Global Fit to Standard with respect to the specific antigen measured. The samples have correlated relative potency values from antigen expression and dsRNA assays: GMP TO's IVRP in dsRNA assay=67; in Ag1 antigen assay=64; in Ag2 antigen assay=64.

### Sample Test Example 2

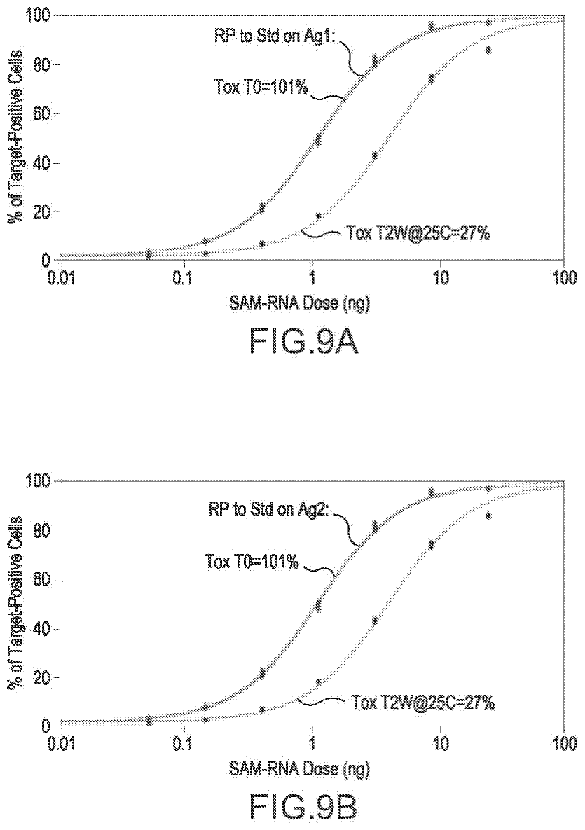

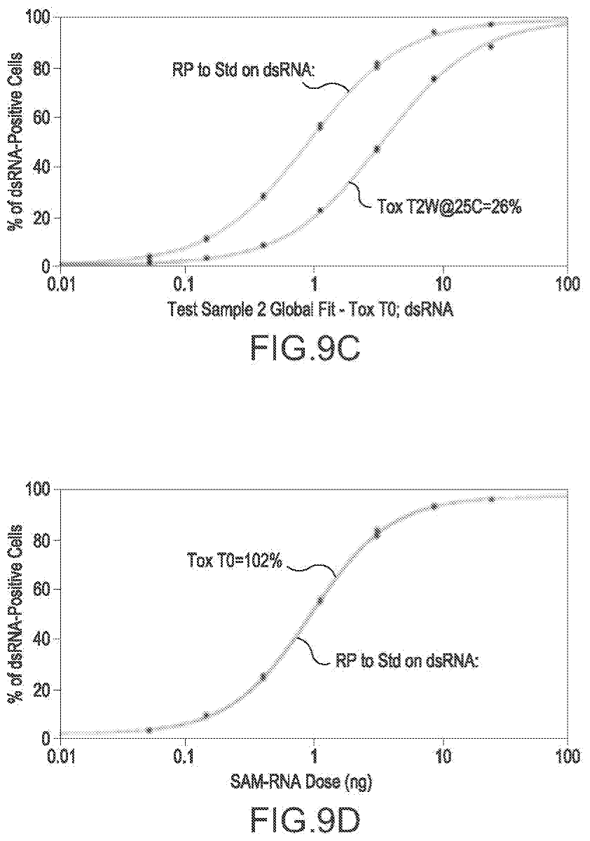

FIGS. 9A-9D are charts showing correlation between antigen and dsRNA expression levels in the evaluation of HSV/LNP product stability, wherein assay results (in terms of % positive cells) are plotted against SAM/RNA dose (ng) Expression levels were measured in the same way as in FIGS. 5A and 5B (flow cytometry, described in Example 1), wherein the assays in each Figure are gE antigen (FIG. 9A) and gI antigen (FIG. 9B) for Tox batch tested at TO time point (T0 Tox) and after storage for 2 weeks at 25° C. (2 weeks at 25° C. Tox), measurement of dsRNA for the sample 2 weeks at 25° C. Tox (FIG. 9C) and T0 Tox (FIG. 9D). The HSV/LNP IVRP (%) to standard values are summarized in the following Table.

| HSVLNP IVRP(%) to Std | dsRNA | Ag1 | Ag2 |
|---|---|---|---|
| T0 GMP | 67 | 64 | 64 |
| T0 Tox | 102 | 101 | 100 |
| T2 W 25 C. Tox | 26 | 27 | 27 |

### Example 3—Summary Showing Correlation Between dsRNA and Antigen Expression for the Different Antigens Used in the Above Examples

As summarized in the below Table, dsRNA and antigen expression were well correlated for the Rabies-LNP, CMV_Ag1-LNP, and HSV-LNP samples tested in the above Examples.

| Sample Name | dsRNA | Antigen |
|---|---|---|
| HSVLNP GMP T0 | 67 | 64 |
| HSVLNP Tox T0 | 102 | 101 |
| HSVLNP Tox T 2 W 25 C. | 26 | 27 |
| CMVLNP N68721-76 Sample1 | 101 | 98 |
| CMVLNP N68721-76 Sample2 | 157 | 158 |
| CMVLNP N68721-76 Sample3 | 158 | 159 |
| CMVLNP N68721-76 Sample4 | 39 | 37 |
| CMVLNP N68721-76 Sample5 | 33 | 31 |
| CMVLNP N68721-65 Sample1 | 98 | 98 |
| CMVLNP N68721-65 Sample2 | 101 | 100 |
| CMVLNP N68721-65 Sample3 | 47 | 46 |
| RabLNP UnMethylated (to FL) | 57 | 52 |
| RabLNP UnCapped (to FL) | N.D. | N.D. |
| RabLNP NoTail (to FL) | 2 | 1 |

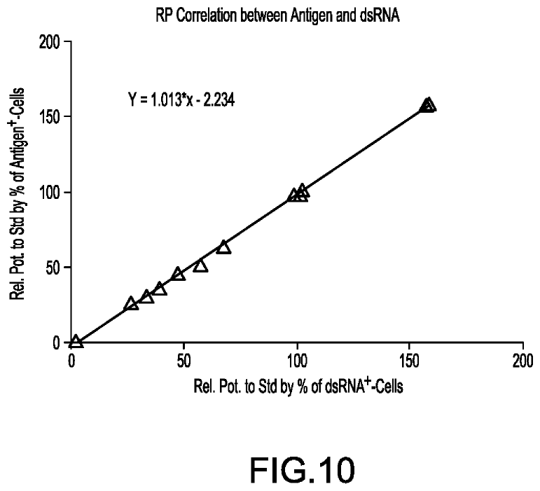

These results are graphically illustrated in FIG. 10 which is a graph showing the relative potency correlation between dsRNA and antigen expression for various different antigens by comparing relative potency to standard by % of antigen positive cells to relative potency to standard by % of dsRNA positive cells.

1. A release assay for determining the potency of a drug product composition comprising self-amplifying mRNA (SAM) that encodes at least one an immunogenic polypeptide or that encodes at least one therapeutic polypeptide and a non-viral delivery system, said method comprising the steps of:
incubating said drug product composition with cells in vitro; and
detecting double-stranded RNA (dsRNA) in the cells.

2. The release assay of claim 1, wherein said SAM encodes at least one immunogenic polypeptide.

3. The release assay of claim 1, wherein said non-viral delivery system comprises lipid nanoparticles.

4. The release assay of claim 1, wherein said detecting step detects a signal that correlates to the amount of dsRNA in said cells.

5. The release assay of claim 1, wherein said detecting step comprises contacting said incubated cells with an antibody that binds dsRNA.

6. The release assay of claim 5, wherein said antibody that is contacted with said cells is a labeled antibody that specifically binds to dsRNA.

7. The release assay of claim 1, wherein said detecting step comprises determining the percentage of said cells that test positive in an assay that measures dsRNA in individual cells.

8. The release assay of claim 7, wherein the percentage of dsRNA positive cells is determined by flow cytometry analysis.

9. The release assay of claim 1, wherein said non-viral delivery system comprises liposomes.

10. The release assay of claim 7, wherein an anti-dsRNA antibody is used to bind to dsRNA in the cytoplasm of said cells and determining the percentage of dsRNA positive cells is performed by flow cytometry analysis.

11. The release assay of claim 1, further comprising the steps of comparing the results of said detecting step with predetermined results to determine if the results of said detecting step fall within pre-determined potency release standards and then releasing the drug product composition if the results of said detecting step fall within said predetermined release standards and not releasing the drug product if the results of said detecting step do not fall within said predetermined potency release standards.

12. The release assay of claim 11, wherein said method is performed on a sample from a lot of said drug product and the lot is not subjected to an assay that measures expression of said polypeptide before releasing the lot.

13. The release assay of claim 1, wherein said delivery system comprises liposomes that encapsulate SAM molecules.

14. The release assay of claim 1, wherein said delivery system comprises a lipid nanoparticle/SAM delivery system where said SAM molecules encode a polypeptide that elicits an immune response against at least one antigen present in a virus selected from the group consisting of rabies virus, HSV, CMV and COVID.

15. The release assay of claim 1, wherein said cells are an established mammalian cell line.

16. The release assay of claim 14, wherein said cells are a BHK cell line or a mouse muscle cell line.

17. The release assay of claim 15, wherein said cells are BHK-12 cells.

18. (canceled)

19. A release assay for checking the potency of a drug product composition comprising self-amplifying mRNA (SAM) that encodes at least one an immunogenic polypeptide and a non-viral delivery system, said assay comprising the steps of:
incubating said drug product composition with cells in vitro;
contacting said incubated cells with a reagent that measures dsRNA;
determining the percentage of said cells that test positive in an assay that measures dsRNA in individual cells; and
checking that the percentage of dsRNA positive cells falls within a predetermined acceptable range.

20. The release assay of claim 19, wherein the reagent is an antibody that binds dsRNA, preferably a labelled antibody that binds dsRNA.

21. The release assay of claim 19, wherein the percentage of dsRNA positive cells is determined by flow cytometry analysis.

22. A process of manufacturing a drug product comprising a composition comprising self-amplifying mRNA (SAM) that encodes at least one an immunogenic polypeptide or that encodes at least one therapeutic polypeptide and a non-viral delivery system for distribution to distributors, medical professionals or patients, the process comprising the steps of:
(a) manufacturing said drug product; and
(b) determining the potency of said drug product by the method of claim 1.

23. The process of claim 22, wherein said method is performed on a sample from a lot of said pharmaceutical product and the lot is not subjected to an assay that measures expression of said polypeptide before releasing the lot.

24. The process of claim 22, further comprising the steps of comparing the results of said detecting step with predetermined results to determine if the results of said detecting step fall within pre-determined potency release standards and then releasing the drug product if the results of said detecting step fall within said predetermined potency release standards and not releasing the drug product if the results of said detecting step do not fall within said predetermined potency release standards.
